(19) United States
(12) Patent Application Publication
(10) Pub. No.: US 2022/0047678 A1
(43) Pub. Date: Feb. 17, 2022

(54) Combination Therapy for Treatment of Liver Disease

(71) Applicants: Gilead Sciences, Inc., Foster City, CA (US); Novo Nordisk A/S, Bagsvaerd (DK)

(72) Inventors: Constantine Stephen Djedjos, Belmont, CA (US); Bryan John McColgan, Stockholm (SE); Robert Paul Myers, Menlo Park, CA (US); Gangadharan Mani Subramanian, Los Altos Hills, CA (US); Lene Melchiorsen, Bagsvaerd (DK); Morten Hansen, Bagsvaerd (DK)

(73) Assignees: Gilead Sciences, Inc., Foster City, CA (US); Novo Nordisk A/S, Bagsvaerd (DK)

(21) Appl. No.: 17/337,576

(22) Filed: Jun. 3, 2021

Related U.S. Application Data
(60) Provisional application No. 63/034,479, filed on Jun. 4, 2020.

(51) Int. Cl. A61K 38/26; A61K 31/519; A61K 31/4439; A61K 9/00; A61P 1/16; A61P 3/04; A61P 3/10

(52) U.S. Cl. CPC A61K 38/26; A61K 31/519; A61K 31/4439; A61P 3/10; A61K 9/0019; A61P 1/16; A61P 3/04; A61K 9/0053

(57) ABSTRACT

The present disclosure relates to a method of treating non-alcoholic steatohepatitis (NASH) comprising administering to a subject with NASH a combination therapy comprising semaglutide, firsocostat, and/or cilofexor.

### CROSS-REFERENCE TO RELATED APPLICATION

This application claims the benefit of priority of U.S. Provisional Application No. 63/034,479, filed Jun. 4, 2020, which is incorporated by reference herein in its entirety for any purpose.

### FIELD

The present disclosure relates to methods of preventing and/or treating liver diseases, such as non-alcoholic steatohepatitis (NASH).

### BACKGROUND

Liver disease is generally classified as acute or chronic based on the duration of the disease. Liver disease may be caused by infection, injury, exposure to drugs or toxic compounds, excessive alcohol use or abuse, impurities in foods, and the abnormal build-up of normal substances in the blood, an autoimmune process, a genetic defect (such as haemochromatosis), or unknown cause(s).

Liver disease is a leading cause of death world-wide. In particular, a diet high in fat damages the liver in ways that are similar to hepatitis. The American Liver Foundation estimates that more than 20 percent of the population has non-alcoholic fatty liver disease (NAFLD). Obesity, unhealthy diets, and sedentary lifestyles may contribute to the high prevalence of NAFLD. NAFLD is considered to cover a spectrum of disease activity and begins as fatty accumulation in the liver (hepatic steatosis). When left untreated, NAFLD can progress to non-alcoholic steatohepatitis (NASH), which has serious adverse effects. Once NASH develops, it causes the liver to swell and scar (i.e. develop cirrhosis) over time.

In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. Genetics has also been known to play a role, as two genetic mutations for this susceptibility have been identified.

Although preliminary reports suggest positive lifestyle changes could prevent or reverse liver damage, there are no effective medical treatments for NAFLD or NASH. Accordingly, there remains a need to provide new effective pharmaceutical agents to treat liver diseases.

### SUMMARY

Disclosed herein are methods of treating and/or preventing non-alcoholic steatohepatitis (NASH) in a subject, comprising administering to the subject (a) semaglutide at a dose of 0.1-3 mg once weekly and (b) firsocostat at a dose of 15-25 mg once daily. In some embodiments, the method comprises administering firsocostat once weekly at a dose selected from 15 mg, 18 mg, 20 mg, 22 mg, and 25 mg. In some embodiments, the method comprises administering firsocostat at a dose of 20 mg once daily. In some embodiments, the method further comprises administering cilofexor at a dose of 20-120 mg once daily. In some embodiments, the method comprises administering cilofexor once daily at a dose selected from 20 mg, 30 mg, 40 mg, 50 mg, 60 mg, 70 mg, 80 mg, 90 mg, 100 mg, 110 mg, and 120 mg. In some embodiments, the method comprises administering cilofexor at a dose of 30 mg once daily. In some embodiments, the method comprises administering cilofexor at a dose of 30 mg and firsocostat at a dose of 20 mg. In some embodiments, the method comprises administering cilofexor at a dose of 100 mg once daily. In some embodiments, the method comprises administering cilofexor at a dose of 100 mg and firsocostat at a dose of 20 mg. In some embodiments, the cilofexor and firsocostat are provided in a combined solid dosage form.

In some embodiments, a method of treating NASH comprises administering to a subject with NASH (a) semaglutide at a dose of 0.1-3 mg once weekly; (b) firsocostat at a dose of 20 mg once daily; and (c) cilofexor at a dose of 30 mg once daily.

In some embodiments, the method comprises administering semaglutide at a dose of 0.24-2.4 mg once weekly. In some embodiments, the method comprises administering semaglutide at an escalating dose from 0.24-2.4 mg once weekly. In some embodiments, the method comprises administering semaglutide once weekly at a dose selected from 0.24 mg, 0.50 mg, 1.0 mg, 1.7 mg, and 2.4 mg. In some embodiments, the method comprises administering semaglutide at a dose of 0.24 mg once weekly for four weeks, followed by a dose of 0.50 mg once weekly for four weeks, followed by a dose of 1.0 mg once weekly for four weeks, followed by a dose of 1.7 mg once weekly for four weeks, followed by a dose of 2.4 mg once weekly for at least four weeks.

In some embodiments, the method comprises treating the subject for at least 24 weeks, at least 36 weeks, at least 48 weeks, at least 60 weeks, at least 72 weeks, at least 84 weeks, at least 96 weeks, or longer. In some embodiments, the method comprises treating the subject for the duration of the subject's life.

Disclosed herein are also methods of treating and/or preventing non-alcoholic steatohepatitis (NASH) in a subject, comprising administering to the subject a) semaglutide at a dose of 0.1-3 mg once weekly; and b) cilofexor at a dose of 20-120 mg once daily. In some embodiments, the method comprises administering semaglutide at a dose of 0.24-2.4 mg once weekly. In some embodiments, the method comprises administering semaglutide at an escalating dose from 0.24-2.4 mg once weekly. In some embodiments, the method comprises administering semaglutide once weekly at a dose selected from 0.24 mg, 0.50 mg, 1.0 mg, 1.7 mg, and 2.4 mg. In some embodiments, the method comprises administering semaglutide at a dose of 0.24 mg once weekly for four weeks, followed by a dose of 0.50 mg once weekly for four weeks, followed by a dose of 1.0 mg once weekly for four weeks, followed by a dose of 1.7 mg once weekly for four weeks, followed by a dose of 2.4 mg once weekly for at least four weeks. In some embodiments, the method comprises administering cilofexor once daily at a dose selected from 20 mg, 30 mg, 40 mg, 50 mg, 60 mg, 70 mg, 80 mg, 90 mg, 100 mg, 110 mg, and 120 mg. In some embodiments, the method comprises administering cilofexor at a dose of 30 mg once daily. In some embodiments, the method comprises administering cilofexor at a dose of 100 mg once daily. In some embodiments, the method comprises treating the subject for at least 24 weeks, at least 36 weeks, at least 48 weeks, at least 60 weeks, at least 72 weeks, at least 84 weeks, at least 96 weeks, or longer. In some embodiments, the method comprises treating the subject for the duration of the subject's life.

In various embodiments, semaglutide is administered by subcutaneous injection. In various embodiments, firsocostat is administered orally. In various embodiments, cilofexor is administered orally.

In some embodiments, the subject showed signs of fibrosis prior to treatment. In some embodiments, the subject has a FibroTest® score of <0.75 prior to treatment. In some embodiments, the subject has ≥10% steatosis prior to treatment. Steatosis may be determined, for example, by MRI-PDFF. In some embodiments, the subject has liver stiffness ≥7 kPa prior to treatment. In some embodiments, liver stiffness is determined by FibroScan®. In some embodiments, the subject was diagnosed with NASH prior to treatment. In some embodiments, the subject was diagnosed with NASH using a liver biopsy prior to treatment. In some embodiments, the subject has type 2 diabetes mellitus.

In various embodiments, the subject has one or more of the following laboratory parameters at a baseline timepoint prior to treatment:

a) alanine aminotransferase (ALT) level≤5× the upper limit of normal (ULN);

b) estimated glomerular filtration rate (eGFR)≥30 mL/min;

c) HbA1c≤9.5%;

d) Serum fructosamine≤381 μmol;

e) INR≤1.2;

f) platelet count≥100,000/μL;

g) total bilirubin<1.3×upper limit of normal (ULN); and/or

h) calcitonin≤100 ng/L.

In some embodiments, eGFR is calculated by the MDRD study equation.

In some embodiments, eGFR is improved following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to eGFR at a baseline timepoint prior to treatment.

In some embodiments, steatosis is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to steatosis at a baseline timepoint prior to treatment. In some embodiments, steatosis is decreased for the duration of the subject's life compared to the subject's steatosis at a baseline timepoint prior to treatment. In some embodiments, steatosis is measured by MRI-PDFF or CAP.

In some embodiments, liver stiffness is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to liver stiffness at a baseline timepoint prior to treatment. In some embodiments, liver stiffness is decreased for the duration of the subject's life compared to the subject's liver stiffness at a baseline timepoint prior to treatment. In some embodiments, liver stiffness is measured by MRE or FibroScan®.

In some embodiments, at least one laboratory parameter selected from ALT, aspartate aminotransferase (AST), bilirubin, gamma-glutamyl transpeptidase (GGT), and alkaline phosphatase (ALP) is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the respective laboratory parameter at a baseline timepoint prior to treatment. In some embodiments, at least one laboratory parameter selected from ALT, aspartate aminotransferase (AST), bilirubin, gamma-glutamyl transpeptidase (GGT), and alkaline phosphatase (ALP) is decreased for the duration of the subject's life compared to the subject's ALT, aspartate aminotransferase (AST), bilirubin, gamma-glutamyl transpeptidase (GGT), and alkaline phosphatase (ALP) at a baseline timepoint prior to treatment.

In some embodiments, the subject's enhanced liver fibrosis (ELF) test score and/or FibroTest® test score is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the respective test score at a baseline timepoint prior to treatment. In some embodiments, enhanced liver fibrosis test score and/or FibroTest® test score is decreased for the duration of the subject's life compared to the subject's enhanced liver fibrosis test score and/or FibroTest® test score at a baseline timepoint prior to treatment.

In some embodiments, the subject's triglyceride level, LDL cholesterol level, total cholesterol level, HbA_{1c}, fasting plasma glucose level, fasting insulin level, and/or HOMA-IR is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the respective test score at a baseline timepoint prior to treatment.

In some embodiments, the subject's body weight is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the subject's body weight at a baseline prior to treatment.

In some embodiments, the subject's FAST score is decreased by at least 0.1, at least 0.2, or at least 0.3 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the FAST score at a baseline timepoint prior to treatment.

### BRIEF DESCRIPTION OF THE DRAWINGS

FIG. 1 shows the structure of semaglutide.

FIG. 2 is a chart summarizing the different treatment groups in the clinical study described in Example 1.

FIG. 3 shows the trial design where CAP means controlled attenuation parameter; LS is liver stiffness; MRE is magnetic resonance elastography; MRI-PDFF is magnetic resonance imaging proton density fat fraction; SEMA is semaglutide; QW is once weekly; and VCTE is vibration-controlled transient elastography.

FIG. 4 shows the trial profile for subjects enrolled in the Example 1 study.

FIG. 5A-C show the change from baseline to week 24 in liver steatosis assessed by MRI-PDFF. The LSₘₑₐₙ change is based on ANCOVA models adjusted for baseline and diabetes status. ANCOVA is analysis of covariance. CI is confidence interval. LSₘₑₐₙ is least squares mean. MRI-PDFF is magnetic resonance imaging proton density fat fraction. FIG. 5A shows the absolute change in liver steatosis. FIG. 5B shows the proportions of patients with a ≥30% and 50% relative reduction in MRI-PDFF. FIG. 5C shows the proportions of patients with normalization of liver fat (<5%) and ≥5% absolute reduction in MRI-PDFF.

FIG. 6A-C show a sensitivity analysis that shows the change from baseline to week 24 in liver steatosis by magnetic resonance imaging proton density fat fraction in patients with imaging data collected within 30 days after the last dose of trial drug. FIG. 6A shows the absolute change from baseline. FIG. 6B shows the proportions of patients with a ≥30% and 50% relative reduction in MRI-PDFF. FIG. 6C shows the proportions of patients with normalization of liver fat (<5%) and ≥5% absolute reduction in MRI-PDFF.

FIG. 7 shows changes in liver steatosis from baseline to week 24 measured by controlled attenuation parameter.

FIG. 8A-D show changes from baseline to week 24 in alanine aminotransferase (FIG. 10A), aspartate aminotransferase (FIG. 10B), liver stiffness by transient elastography (FIG. 10C) and FibroScan-AST (FAST) score (FIG. 10D).

FIG. 9 shows the proportions of patients with a ≥25% relative reduction in liver stiffness by transient elastography.

FIG. 10 is a chart summarizing the different treatment groups in the clinical study described in Example 2.

### DETAILED DESCRIPTION

### Definitions and General Parameters

As used in the present specification, the following terms and phrases are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

In the human liver, UDP-glucuronosyltransferases act on certain compounds having amino, carbamyl, thio (sulfhydryl) or hydroxyl groups to conjugate uridine diphosphate-α-D-glucuronic acid through glycoside bonds, or to esterify compounds with carboxy or hydroxyl groups in the process of phase II metabolism. Compounds of the present disclosure may be glucuronidated, that is to say, conjugated to glucuronic acid, to form glucuronides, particularly (β-D)glucuronides.

One step in the formation of bile is the conjugation of the individual bile acids with an amino acid, particularly glycine or taurine. Compounds of the present disclosure may be conjugated with glycine or taurine at a substitutable position.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e. groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to persons skilled in the art.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts.

The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, Jan. 1977 vol. 66, No. 1, and other sources.

Furthermore, compounds disclosed herein may be subject to tautomerism. Where tautomerism, e.g. keto-enol tautomerism, of compounds or their prodrugs may occur, the individual forms, like e.g. the keto and enol form, are each within the scope of the disclosure as well as their mixtures in any ratio. The same applies for stereoisomers, like e.g. enantiomers, cis/trans isomers, diastereomers, conformers and the like.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

In certain embodiments, provided are optical isomers, racemates, or other mixtures thereof of the compounds described herein or a pharmaceutically acceptable salt or a mixture thereof. If desired, isomers can be separated by methods well known in the art, e.g. by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are non-superimposable mirror images of one another. “Diastereomers” are stereoisomers that have at least two asymmetric atoms, but which are not mirror-images of each other.

Compounds disclosed herein and their pharmaceutically acceptable salts may, in some embodiments, include an asymmetric center and may thus give rise to enantiomers, diastereomers, and other stereoisomeric forms that may be defined, in terms of absolute stereochemistry, as (R) or (S) or, as (D) or (L) for amino acids. Some embodiments include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorus, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds disclosed herein, in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have beneficial DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An 18F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt thereof for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### Liver Diseases

Liver diseases may involve acute or chronic damage to the liver depending on the cause and severity of the condition. The liver damage may be induced by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, non-alcoholic fatty liver disease (NAFLD) including non-alcoholic steatohepatitis (NASH), alcoholic steatohepatitis (ASH), primary biliary cirrhosis (PBC), primary sclerosing cholangitis (PSC), and hepatitis, including both viral and alcoholic hepatitis.

NAFLD is characterized by excessive accumulation of fat in hepatocytes and is often associated with features of metabolic syndrome (e.g. type 2 diabetes mellitus, insulin resistance, hyperlipidemia, and hypertension). The occurrence of NAFLD is increasing due to increasing frequency of obesity. If left untreated, NAFLD may progress to non-alcoholic steatohepatitis (NASH), where inflammation develops in the steatotic liver potentially leading to fibrosis formation. A subset (˜20%) of NAFLD patients develop NASH.

NASH is characterized histologically by steatosis, ballooning of hepatocytes, and inflammation, which, may lead to hepatic scarring (i.e. fibrosis). Patients diagnosed with NASH may progress to advanced stage liver fibrosis and eventually cirrhosis with liver failure, which, potentially may require liver transplantation. NASH has become one of the major causes of end stage liver disease and cirrhosis.

### Firsocostat

Firsocostat is an acetyl coA carboxylase (ACC) inhibitor having the structure of Formula (I):

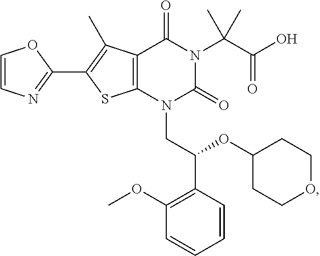 [SMILES: COc1ccccc1[C@H](Cn1c(=O)n(C(C)(C)C(=O)O)c(=O)c2c(C)c(-c3ncco3)sc21)OC1CCOCC1]

or a pharmaceutically acceptable salt thereof.

As used herein, and in the absence of a specific reference to a particular pharmaceutically acceptable salt and/or solvate of firsocostat, any dosages, whether expressed in e.g. milligrams or as a % by weight, should be taken as referring to the amount of firsocostat, i.e. the amount of:

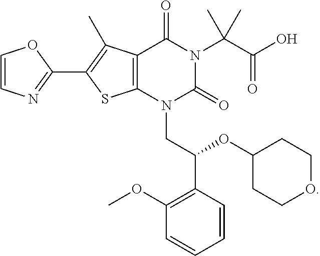 [SMILES: COc1ccccc1[C@H](Cn1c(=O)n(C(C)(C)C(=O)O)c(=O)c2c(C)c(-c3ncco3)sc21)OC1CCOCC1]

For example, therefore, a reference to “25 mg firsocostat or a pharmaceutically acceptable salt and/or solvate thereof” means an amount of firsocostat or a pharmaceutically acceptable salt and/or solvate thereof which provides the same amount of firsocostat as 25 mg of firsocostat free acid.

The amount of firsocostat in a solid oral dosage form provided herein is generally about 10 mg to about 30 mg, for instance about 15 mg to about 25 mg, and more typically about 18 mg to about 22 mg. In some embodiments, the amount of firsocostat in a solid oral dosage form provided herein is generally between 10 mg and 30 mg, for instance within the range of 15 mg to 25 mg, and more typically between 18 mg and 22 mg. In some embodiments, the amount of firsocostat in a solid oral dosage form provided herein is 20 mg.

In some embodiments, the firsocostat is provided in a combination product further comprising cilofexor. In some embodiments, the combination product a solid oral dosage form.

The compound of Formula (I) may be synthesized and characterized using methods known to those of skill in the art, such as those described in PCT Publication No. WO 2013/071169 (compound I-246; see also U.S. Publication No. 2013/0123231).

ACC catalyzes the ATP-dependent carboxylation of acetyl-CoA to form malonyl-CoA. This reaction, which proceeds in two half-reactions, a biotin carboxylase (BC) reaction and a carboxyltransferase (CT) reaction, is the first committed step in fatty acid (FA) biosynthesis and is the rate-limiting reaction for the pathway. In addition to its role as a substrate in FA biosynthesis, malonyl-CoA (the product of the ACC-catalyzed reaction) also plays an important regulatory role in controlling mitochondrial FA uptake through allosteric inhibition of carnitine palmitoyltransferase I (CPT-I), the enzyme that catalyzes the first committed step in mitochondrial FA oxidation. Malonyl-CoA is therefore a key metabolic signal for the control of FA production and utilization in response to dietary changes and altered nutritional requirements in animals, for example during exercise, and plays a key role in controlling the switch between carbohydrate and fat utilization in liver and skeletal muscle (Harwood, Expert Opin Ther Targets, 2005, 9: 267-281).

An “ACC inhibitor” refers to an agent that is capable of binding and inhibiting ACC. ACC inhibitors may act as inhibitors or partial inhibitors of ACC. The activity of an ACC inhibitor may be measured by methods known in the art, such as those described and cited in U.S. Pat. No. 8,969,557, and/or in U.S. Patent Publication No. 2016/0108061.

### Cilofexor

Cilofexor is a farnesoid X-activated receptor (FXR) agonist having the structure of Formula (II):

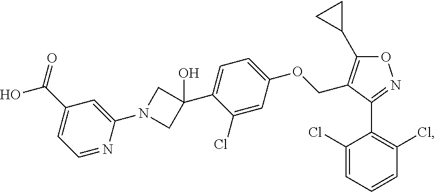 [SMILES: O=C(O)c1ccnc(N2CC(O)(c3ccc(OCc4c(-c5c(Cl)cccc5Cl)noc4C4CC4)cc3Cl)C2)c1]

or a pharmaceutically acceptable salt thereof.

As used herein, and in the absence of a specific reference to a particular pharmaceutically acceptable salt and/or solvate of cilofexor, any dosages, whether expressed in e.g. milligrams or as a % by weight, should be taken as referring to the amount of cilofexor, i.e. the amount of:

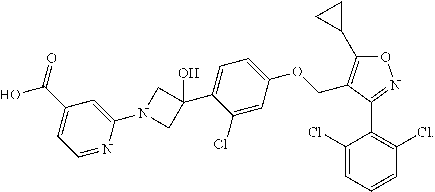 [SMILES: O=C(O)c1ccnc(N2CC(O)(c3ccc(OCc4c(-c5c(Cl)cccc5Cl)noc4C4CC4)cc3Cl)C2)c1]

For example, therefore, a reference to “25 mg cilofexor or a pharmaceutically acceptable salt and/or solvate thereof” means an amount of cilofexor or a pharmaceutically acceptable salt and/or solvate thereof which provides the same amount of cilofexor as 25 mg of cilofexor free acid.

The amount of cilofexor in a solid oral dosage form provided herein is generally about 10 mg to about 200 mg, for instance about 20 mg to about 150 mg, and more typically about 25 mg to about 35 mg or about 90 mg to about 110 mg. In some embodiments, the amount of cilofexor in a solid oral dosage form provided herein is generally between 10 mg and 200 mg, for instance within the range of 20 mg to 150 mg, and more typically between 25 mg and 35 mg or between 90 mg and 110 mg. In some embodiments, the amount of cilofexor in a solid oral dosage form provided herein is 30 mg or 100 mg. In certain embodiments, the solid oral dosage form contains 30 mg cilofexor e.g. as about 36.2 mg of cilofexor tromethamine salt. In certain embodiments, the solid oral dosage form contains 100 mg cilofexor e.g. as about 120.6 mg of cilofexor tromethamine salt.

In some embodiments, the cilofexor is provided in a combination product further comprising firsocostat. In some embodiments, the combination product a solid oral dosage form.

The compound of Formula (II) may be synthesized and characterized using methods known to those of skill in the art, such as those described in U.S. Publication No. 2014/0221659.

FXR, also often referred to as NR1H4 (nuclear receptor subfamily 1, group H, member 4) when referring to the human receptor, is a nuclear hormone receptor. FXR has been associated with multiple biological functions. FXR is primarily expressed in the liver and throughout the entire gastrointestinal tract, but is also found in the kidney, adrenal gland, and ovary. FXR is associated with controlling intracellular gene expression and may be involved in paracrine and endocrine signaling. In the intestine and liver, FXR functions as a regulator of bile acid homeostasis and hepatic lipogenesis. FXR has also been associated with Kupffer cells and liver sinusoidal endothelial cells of the liver, wherein it is believed to have functions related to inflammation, fibrosis, and portal hypertension.

An “FXR agonist” refers to any agent that is capable of binding and activating FXR. FXR agonists may act as agonists or partial agonists of FXR. The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

### Cilofexor/Firsocostat Combination Products

The amount of firsocostat in a solid oral dosage form comprising a combination of firsocostat and cilofexor provided herein is generally between 10 mg and 30 mg, for instance within the range of 15 mg to 25 mg, and more typically between 18 mg and 22 mg. In some embodiments, the amount of firsocostat in a combination product solid oral dosage form comprising a combination of firsocostat and cilofexor provided herein is 20 mg.

The amount of cilofexor in a solid oral dosage form comprising a combination of firsocostat and cilofexor provided herein is generally between 10 mg and 200 mg, for instance within the range of 20 mg to 150 mg, and more typically between 25 mg and 35 mg or between 90 mg and 110 mg. In some embodiments, the amount of cilofexor in a solid oral dosage form comprising a combination of firsocostat and cilofexor provided herein is 30 mg or 100 mg. In certain embodiments, the solid oral dosage form contains 30 mg cilofexor e.g. as about 36.2 mg of cilofexor tromethamine salt. In certain embodiments, the solid oral dosage form contains 100 mg cilofexor e.g. as about 120.6 mg of cilofexor tromethamine salt.

In some embodiments, the solid oral dosage form contains a dose of 30 mg and firsocostat at a dose of 20 mg. In some embodiments, the solid oral dosage form contains a dose of 100 mg and firsocostat at a dose of 20 mg.

### Semaglutide

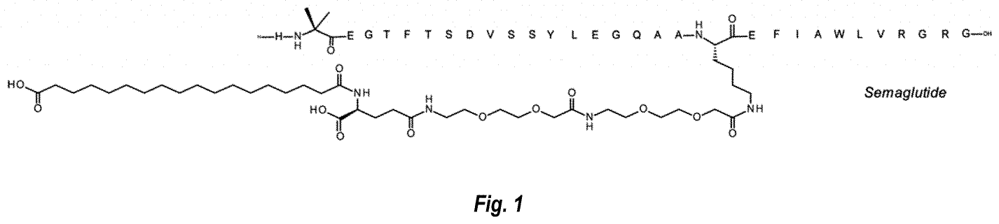

Semaglutide is a glucagon-like peptide-1 (GLP-1) receptor agonist (or GLP-1 analog) having the structure as shown in FIG. 1. See Lau et al., J. Med. Chem. 2015, 58: 1370-7380. Semaglutide may be prepared using methods known to those skilled in the art, such as those described in U.S. Pat. No. 8,129,343.

Glucagon-like peptide-1 (GLP-1) is a 37 amino acid peptide that is secreted by intestinal L-cells and released into the body's circulation in response to food ingestion. The plasma concentration of GLP-1 rises from a fasting level of approximately 15 μmol/L to a peak postprandial level of 40 μmol/L. In addition to the insulinotropic effect, GLP-1 suppresses glucagon secretion, delays gastric emptying (Wettergren A., et al., Dig Dis Sci 1993, 38:665-73) and may enhance peripheral glucose disposal (D'Alessio, D. A. et al., J. Clin Invest 1994, 93:2293-6). GLP-1 peptides have fast clearance and short half-lives. Thus, therapeutic GLP-1 analogs, such as semaglutide, have been developed to lengthen its duration in vivo. Semaglutide is currently FDA approved and marketed as Ozempic® for the treatment of type 2 diabetes.

### Methods of Treating Liver Disease

Provided herein are methods of treating and/or preventing nonalcoholic steatohepatitis (NASH) in a patient in need thereof, comprising administering to the patient a) a therapeutically effective amount of firsocostat and a therapeutically effective amount of semaglutide, b) a therapeutically effective amount of cilofexor and a therapeutically effective amount of semaglutide, or c) a therapeutically effective amount of firsocostat, a therapeutically effective amount of cilofexor and a therapeutically effective amount of semaglutide.

In some embodiments, a method of treating and/or preventing NASH comprises administering semaglutide at a dose of 0.1-3 mg once weekly and administering firsocostat at a dose of 15-25 mg once daily. In some embodiments, semaglutide is administered at a dose of 0.24-2.4 mg once weekly, such as an escalating dose from 0.24-2.4 mg once weekly. In some embodiments, the method comprises administering semaglutide once weekly at a dose selected from 0.24 mg, 0.50 mg, 1.0 mg, 1.7 mg, and 2.4 mg. The escalating dose may be, for example, a dose of 0.24 mg for four weeks, followed by a dose of 0.50 mg for four weeks, followed by a dose of 1.0 mg for four weeks, followed by a dose of 1.7 mg for four weeks, followed by a dose of 2.4 mg for at least four weeks. In some embodiments, the method comprises administering firsocostat once weekly at a dose selected from 15 mg, 18 mg, 20 mg, 22 mg, and 25 mg. In some embodiments, firsocostat is administered at a dose of 20 mg once daily. In various embodiments, the method further comprises administering cilofexor at a dose of 20-120 mg once daily, such as at a dose of 30 mg once daily or a dose of 100 mg once daily. In some embodiments, the method comprises administering cilofexor once daily at a dose selected from 20 mg, 30 mg, 40 mg, 50 mg, 60 mg, 70 mg, 80 mg, 90 mg, 100 mg, 110 mg, and 120 mg.

In some embodiments, a method of treating and/or preventing NASH comprises administering semaglutide at a dose of 0.1-3 mg once weekly and administering cilofexor at a dose of 20-120 mg once daily. In some embodiments, semaglutide is administered at a dose of 0.24-2.4 mg once weekly, such as an escalating dose from 0.24-2.4 mg once weekly. In some embodiments, the method comprises administering semaglutide once weekly at a dose selected from 0.24 mg, 0.50 mg, 1.0 mg, 1.7 mg, and 2.4 mg. The escalating dose may be, for example, a dose of 0.24 mg for four weeks, followed by a dose of 0.50 mg for four weeks, followed by a dose of 1.0 mg for four weeks, followed by a dose of 1.7 mg for four weeks, followed by a dose of 2.4 mg for at least four weeks. In some embodiments, cilofexor is administered at a dose of 20-120 mg once daily, such as at a dose of 30 mg once daily or a dose of 100 mg once daily.

In some embodiments, a method of treating and/or preventing NASH comprises administering to a subject with NASH in need of such treatment semaglutide at a dose of 0.1-3 mg once weekly and administering cilofexor at a dose of 20-120 mg once daily and administering firsocostat at a dose of 15-25 mg once daily. In some embodiments, semaglutide is administered at a dose of 0.24-2.4 mg once weekly, such as an escalating dose from 0.24-2.4 mg once weekly. In some embodiments, the method comprises administering semaglutide once weekly at a dose selected from 0.24 mg, 0.50 mg, 1.0 mg, 1.7 mg, and 2.4 mg. The escalating dose may be, for example, a dose of 0.24 mg for four weeks, followed by a dose of 0.50 mg for four weeks, followed by a dose of 1.0 mg for four weeks, followed by a dose of 1.7 mg for four weeks, followed by a dose of 2.4 mg for at least four weeks. In some embodiments, the method comprises administering firsocostat once weekly at a dose selected from 15 mg, 18 mg, 20 mg, 22 mg, and 25 mg. In some embodiments, cilofexor is administered at a dose of 20-120 mg once daily, such as at a dose of 30 mg once daily or a dose of 100 mg once daily. In some embodiments, the method comprises administering cilofexor once daily at a dose selected from 20 mg, 30 mg, 40 mg, 50 mg, 60 mg, 70 mg, 80 mg, 90 mg, 100 mg, 110 mg, and 120 mg.

In various embodiments, semaglutide is administered by injection, such as subcutaneous injection. In various embodiments, cilofexor and/or firsocostat are administered orally.

In various embodiments, cilofexor and firsocostat are administered orally in a fixed-dose combination product.

The presence of active liver disease, such as NASH, can be detected by a variety of laboratory parameters. For example, alanine aminotransferase (ALT) and aspartate aminotransferase (AST) levels above clinically accepted normal ranges are known to be indicative of on-going liver damage. Routine monitoring of liver disease patients for blood levels of ALT and AST is used clinically to measure progress of the liver disease while on medical treatment. Reduction of elevated ALT and AST to within the accepted normal range is taken as clinical evidence of a reduction in the severity of the patient's liver damage. Additional blood parameters indicating the presence of liver disease include estimated glomerular filtration rate (eGFR) that is lower than a normal range (in some embodiments, a normal range is 90 or higher, or 80 or higher, or 70 or higher, or 60 and higher); hemoglobin A1c (HbA1C) that is higher than a normal range (in some embodiments, a normal range is between 4% and 6%); serum fructosamine that is higher than a normal range (in some embodiments, a normal range is 200-285 μmol/L when serum albumin is 5 g/dL); prothrombin time (PT), which is expressed as international normalized ratio (INR), that is higher than normal (in some embodiments, a normal INR is less than or equal to 1.2); platelet count that is lower than a normal range (in some embodiments, a normal range is 150,000-450,000 platelets/μL), total bilirubin that is higher than a normal range (in some embodiments, a normal range is 0.2-1.2 mg/dL, or 0.2-1.9 mg/dL), and calcitonin that is higher than a normal range (in some embodiments, a normal range is less than 5 pg/mL in a female and less than 8.4 pg/mL in a male). Other indicators of liver disease include cirrhosis, fibrosis and fibrogenesis.

In some embodiments, the methods provided herein decrease steatosis, decrease liver stiffness, decrease liver fibrosis, improve eGFR, and/or decrease one or more laboratory parameters selected from alanine aminotransferase (ALT), aspartate aminotransferase (AST), bilirubin, gamma-glutamyl transpeptidase (GGT), and alkaline phosphatase (ALP). In some embodiments, the methods provided herein decrease a subject's triglyceride level, LDL cholesterol level, and/or total cholesterol level. In various embodiments, the decrease is observed following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer. In some embodiments, the decrease is observed following treatment for the duration of the subject's life.

Methods of measuring steatosis in the liver are known in the art, and include, for example, MRI-PDFF and controlled attenuation parameter (CAP) score using, for example, FibroScan®. FibroScan® is a non-invasive test that uses ultrasound to determine the degree of scarring (fibrosis) and steatosis in the liver. In some embodiments, the subject has ≥5% steatosis prior to treatment, for example, as determined by MRI-PDFF. In some embodiments, the subject has ≥10% steatosis prior to treatment, for example, as determined by MRI-PDFF. In some embodiments, the subject has a CAP score of >215 prior to treatment. In some embodiments, steatosis is decreased by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, or at least about 95% following the treatment provided herein. In some embodiments, steatosis is decreased by at least about 5%, at least about 10%, or at least about 20% following the treatment provided herein for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to steatosis at a baseline timepoint prior to treatment. In some embodiments, following treatment, a subject's CAP score is reduced by at least 5 dB/m, at least 10 dB/m, at least 15 dB/m, at least 20 dB/m, at least 25 dB/m, at least 30 dB/m, at least 35 dB/m, at least 35 dB/m, at least 40 dB/m, at least 45 dB/m, at least 50 dB/m, at least 55 dB/m, or at least 60 dB/m. In some embodiments, following treatment, a subject's CAP score is less than 300, less than 290, less than 280, less than 270, less than 260, less than 250, less than 240, less than 230, or less than 220. In some embodiments, following treatment, a subject's CAP score is equal to or less than 215. In some embodiments, following treatment, the median relative MRI-PDFF is reduced by at least 30%, at least 40%, or at least 50% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to MRI-PDFF at a baseline timepoint prior to treatment.

Methods of measuring liver stiffness are known in the art and include, for example, magnetic resonance elastography (MRE) and FibroScan®. In some embodiments, a subject has liver stiffness ≥7 kPa prior to treatment. In some embodiments, liver stiffness is decreased by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, or at least about 95% following the treatment provided herein. In some embodiments, liver stiffness is decreased by at least 25% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to liver stiffness at a baseline timepoint prior to treatment. In some embodiments, following treatment, a subject's liver stiffness is less than 25 kPa, less than 20 kPa, less than 15 kPa, less than 10 kPa, or less than 7 kPa. In some embodiments, following treatment, a subject's liver stiffness is reduced by at least 2 kPa, at least 3 kPa, at least 4 kPa, at least 5 kPa, at least 6 kPa, at least 7 kPa, at least 8 kPa, at least 9 kPa, or at least 10 kPa.

Methods of measuring liver fibrosis are known in the art, and include, for example, enhanced liver fibrosis (ELF) test and its components (including, for example, TIMP metallopeptidase inhibitor 1 (TIMP1), procollagen III N-terminal propeptide (PIII-NP), and hyaluronic acid), FibroScan®, and FibroTest® (also referred to as FibroSure®). FibroSure® is a serum biomarker test that is designed to assess liver fibrosis in patients with chronic viral hepatitis B or C, alcoholic liver disease, and metabolic steatohepatitis (for those who are overweight, have diabetes, or hyperlipidemia). In some embodiments, a subject has evidence of fibrosis prior to treatment. In some embodiments, a subject has a fibrosis score of F2 or higher prior to treatment, as determined by FibroScan®. In some embodiments, a subject has a fibrosis score of F3 or F4 prior to treatment, as determined by FibroScan®. In some embodiments, liver fibrosis is decreased by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, or at least about 95% following the treatment provided herein. In some embodiments, liver fibrosis is decreased by at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the respective test score at a baseline timepoint prior to treatment. In some embodiments, following treatment, a subject's fibrosis score is reduced by one level, or by two levels, or by three levels, following treatment. In some embodiments, a subject's fibrosis score is reduced from F2 to F1 or F0 following treatment. In some embodiments, a subject's fibrosis score is reduced from F3 to F2, F1, or F0 following treatment. In some embodiments, a subject's fibrosis score is reduced from F4 to F3, F2, F1, or F0 following treatment. In some embodiments, a subject's liver fibrosis is determined using an enhanced liver fibrosis (ELF) test score or FibroTest® test score, and the ELF test score is reduced by at least 0.3, at least 0.4, or at least 0.5 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to ELF test score at a baseline timepoint prior to treatment.

Methods of measuring various clinical markers of liver disease, such as alanine aminotransferase (ALT), aspartate aminotransferase (AST), bilirubin, gamma-glutamyl transpeptidase (GGT), alkaline phosphatase (ALP), triglycerides, LDL cholesterol, and total cholesterol, are known in the art. In some embodiments, a subject has an alanine aminotransferase (ALT) level≤5× the upper limit of normal (ULN) prior to treatment. In some embodiments, a subject has a bilirubin level≤1.3× the ULN prior to treatment. ULN for clinical markers may be determined based on reference populations. In some embodiments, the level of one or more clinical markers of liver disease is decreased by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, or at least about 95% following the treatment provided herein. In some embodiments, ALT is reduced by at least 10 U/L, at least 20 U/L, or at least 30 U/L; AST is reduced by at least 10 U/L or at least 20 U/L; GGT is reduced by at least 10 U/L, at least 20 U/L, or at least 30 U/L; and/or ALP is reduced by at least 5 U/L or at least 10 U/L; following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective laboratory parameter at a baseline timepoint prior to treatment.

Methods of measuring estimated glomerular filtration rate (eGFR) are known in the art, and include, for example, measuring creatinine clearance and calculating eGFR using the Modification of Diet in Renal Disease (MDRD) Study equation:

eGFR(mL/min/1.73 m²)=175×Serum Creatinine^{-1.154}×(Age)^{-0.203}×(1.212 if African American)×(0.742 if female).

In some embodiments, the subject has an eGFR of ≥30 mL/min but less than 60 mL/min prior to treatment. In some embodiments, the methods provided herein improve eGFR. In various embodiments, the improvement is observed following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer. In various embodiments, the improvement is observed following treatment for the duration of the subject's life. In some embodiments, the subject's eGFR improves by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, or at least about 95% following the treatment provided herein. In some embodiments, following treatment, a subject's eGFR is at least 40 mL/min, at least 50 mL/min, at least 60 mL/min, at least 70 mL/min, at least 80 mL/min, at least 90 mL/min, or at least 100 mL/min. In some embodiments, following treatment, a subject's eGFR improves by at least 10 mL/min, at least 15 mL/min, at least 20 mL/min, at least 25 mL/min, or at least 30 mL/min. In some embodiments, a subject's eGFR is improved by at least 20 mL/min following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the subject's eGFR at a baseline timepoint prior to treatment

The FibroScan-AST (FAST) score combines liver stiffness measured by TE, steatosis by CAP, and serum AST for the non-invasive identification of patients with NASH and ≥F2 fibrosis. In some embodiments, the subject's FAST score is decreased by at least 0.1, at least 0.2, or at least 0.3 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the FAST score at a baseline timepoint prior to treatment.

In some embodiments, the subject's body weight is decreased by at least 5% following treatment for at least 24 weeks, at least 36 weeks, at least 48 weeks, at least 60 weeks, at least 72 weeks, at least 84 weeks, at least 96 weeks, or longer, compared to the respective test score at a baseline timepoint prior to treatment.

It has been observed that patients having NASH are on average about 2.8 years older than healthy patients in epigenetic testing. Thus, in one embodiment, the disclosed methods for the treatment of NASH would be useful for slowing, improving or reversing epigenetic age or effects of aging due to NASH. In another embodiment, the disclosed methods may be useful for improvement or reversal of aging effects due to NASH.

### Pharmaceutical Compositions

In some embodiments, the method includes administering injectable semaglutide. Semaglutide solution for injection is a colorless or almost colorless liquid, free from turbidity and essentially free from particulate matter, and has the composition shown in Table 1.

TABLE 1

| | | |
|---|---|---|
| Ingredient | Function | Pharmacopoeia |

Drug Substance

| | | |
|---|---|---|
| Semaglutide | Active ingredient | Novo Nordisk A/S |

Other Ingredients

| | | |
|---|---|---|
| Disodium hydrogen | Buffering agent | USP/Ph. Eur |
| phosphate, dihydrate | | |
| Propylene glycol | Isotonic agent | USP/JP/Ph. Eur |
| Phenol | Preservative | USP/JP/Ph. Eur |
| HCl | pH adjustment | USP/JP/Ph. Eur |
| NaOH | pH adjustment | USP/JP/Ph. Eur |
| Water for injection | solvent | USP/JP/Ph. Eur |

In some embodiments, firsocostat and/or cilofexor is administered orally.

In some embodiments, firsocostat may be administered as 20 mg tablets. In addition to the active ingredient, firsocostat tablets may contain one or more of the following inactive ingredients: lactose monohydrate, microcrystalline cellulose, croscarmellose sodium, magnesium stearate, polyvinyl alcohol, titanium dioxide, polyethylene glycol, and talc. In some embodiments, the firsocostat tablets may contain lactose monohydrate, microcrystalline cellulose, croscarmellose sodium, magnesium stearate, polyvinyl alcohol, titanium dioxide, polyethylene glycol, and talc as inactive ingredients.

In some embodiments, cilofexor may be administered as 100 mg and/or 30 mg (as free form equivalent) tablets. The tablets may contain cilofexor (tromethamine salt), one or more inactive ingredients including mannitol, microcrystalline cellulose, crospovidone, magnesium stearate and one or more film-coating material including polyvinyl alcohol, polyethylene glycol, titanium dioxide, talc, yellow iron oxide and black iron oxide. In some embodiments, the cilofexor tablets may contain mannitol, microcrystalline cellulose, crospovidone, and magnesium stearate as inactive ingredients and polyvinyl alcohol, polyethylene glycol, titanium dioxide, talc, yellow iron oxide and black iron oxide as film-coating materials.

In some embodiments, cilofexor and firsocostat may be administered as cilofexor/firsocostat 30 mg/20 mg tablets that are a fixed-dose combination product containing 30 mg of cilofexor (free-form equivalent) and 20 mg of firsocostat, and one or more inactive ingredients including mannitol, microcrystalline cellulose, crospovidone, magnesium stearate and one or more film-coating material including polyvinyl alcohol, polyethylene glycol, titanium dioxide, talc, yellow iron oxide and red iron oxide.

Pharmaceutical compositions for the drugs provided herein may be in a form suitable for the administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In various embodiments, for oral use, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure may contain the active materials in admixture with excipients suitable for the manufacture of aqueous suspensions. Such excipients include a suspending agent, such as, for example, sodium carboxymethylcellulose, methylcellulose, hydroxypropyl methylcellulose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacinth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that these examples are exemplary and not exhaustive. Many changes can be made in the specific embodiments which are disclosed and still obtain a like or similar result without departing from the spirit and scope of the disclosure.

### Example 1: Clinical Study Evaluating Combination Therapy in Subjects with NASH

### A. Overview of Study Design

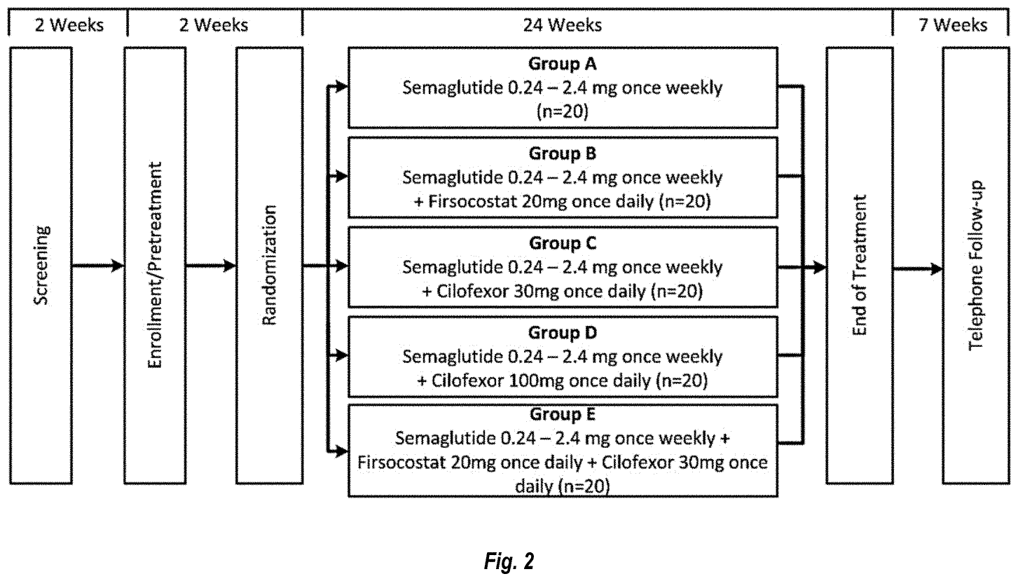

This is a proof of concept, open-label study evaluating the safety, tolerability, and efficacy of monotherapy and combination regimens in subjects with NASH. Subjects meeting the study's entry criteria are randomly assigned in a 1:1:1:1:1 ratio to 1 of 5 treatment groups, with approximately 20 subjects in each group, as shown in the FIG. 2. Randomization is stratified by the presence or absence of type 2 diabetes mellitus, as determined by medical history or based on the Screening laboratory values if previously undiagnosed (hemoglobin A1c [HbA1c]≥6.5%). The study may last up to 35 weeks, which includes a 2-week Screening period, a 2 week Pre-Treatment period, up to a 24-week Treatment period, and a 7-week Follow-Up period.

### B. Subject Population

This study enrolled approximately 100 subjects with a clinical diagnosis of nonalcoholic fatty liver disease (NAFLD) with a Screening FibroTest®<0.75 (unless all historical liver biopsies do not reveal cirrhosis), a Screening MRI-PDFF with ≥10% steatosis (as assessed by the central reader), and a Screening FibroScan® with liver stiffness ≥7 kPa, or subjects with a historical liver biopsy within 6 months of the date of the Screening Visit consistent with NASH (defined as the presence of steatosis, inflammation, and ballooning) with stage 2-3 fibrosis according to the NASH Clinical Research Network (CRN) classification (or equivalent).

### Inclusion Criteria

Subjects must meet all of the following inclusion criteria to participate in the study.
1) Men and women between 18-75 years of age; inclusive based on the date of the Screening Visit;
2) Subjects must meet all of the following conditions:
   a) Clinical diagnosis of nonalcoholic fatty liver disease (NAFLD);
   b) Screening FibroTest®<0.75, unless all previous historical liver biopsies do not reveal cirrhosis. In subjects with Gilbert's syndrome or hemolysis, FibroTest® is calculated using direct bilirubin instead of total bilirubin;
   c) Screening MRI-PDFF with >10% steatosis, as assessed by the central reader. Historical MRI-PDFF within 4 weeks of the date of the Screening Visit may be used if deemed acceptable by the central reader;
   d) Screening FibroScan® with liver stiffness ≥7 kPa. Historical FibroScan® within 4 weeks of the date of the Screening Visit is acceptable;
      OR
   e) A historical liver biopsy within 6 months of the date of the Screening Visit consistent with NASH (defined as the presence of steatosis, inflammation, and ballooning) with stage 2-3 fibrosis according to the NASH Clinical Research Network (CRN) classification (or equivalent), as reviewed by the Medical Monitor;
3) Subject has the following laboratory parameters at the Screening Visit, as determined by the central laboratory:
   a) Alanine aminotransferase (ALT)≤5×ULN;
   b) eGFR≥30 milliliter/minute (mL/min), as calculated by the MDRD study equation;
   c) HbA1c≤9.5% (or serum fructosamine 381 μmol if HbA1c is unable to be resulted);
   d) INR≤1.2, unless due to therapeutic anti-coagulation therapy;
   e) Platelet count≥100,000/μL;
   f) Total bilirubin<1.3×ULN unless alternate etiology such as Gilbert's syndrome present;
   g) Calcitonin≤100 ng/L;
4) Body Mass Index (BMI)>23 kg/m2 and body weight of >60 kg;
5) A negative serum pregnancy test is required for female subjects of childbearing potential.

### Exclusion Criteria

Subjects who meet any of the following criteria are excluded from the study:
1) Documented weight loss >5% within 6 months of the date of the Screening Visit;
2) Any historical liver biopsy consistent with cirrhosis;
3) Alcohol consumption greater than 21 oz/week for males or 14 oz/week for females (1 oz/30 mL of alcohol is present in 1 12 oz/360 mL beer, 1 4 oz/120 mL glass of wine, and a 1 oz/30 mL measure of 40% proof alcohol);
4) Any history of decompensated liver disease, including ascites, hepatic encephalopathy, or variceal bleeding;
5) Other causes of liver disease, including but not limited to: alcoholic liver disease, hepatitis B, hepatitis C, autoimmune disorders (e.g., primary biliary cholangitis [PBC], primary sclerosing cholangitis [PSC], autoimmune hepatitis), drug-induced hepatotoxicity, Wilson disease, clinically significant iron overload, or alpha-1-antitryspin deficiency requiring treatment;
6) History of liver transplantation;
7) History of hepatocellular carcinoma;
8) Weight reduction surgery in the past 2 years or planned during the study;
9) Chronic hepatitis B (HBsAg positive);
10) Chronic hepatitis C (HCV RNA positive). Subjects cured of HCV infection less than 2 years prior to the date of the Screening Visit are not eligible;
11) HIV Ab positive;
12) Unstable cardiovascular disease as defined by any of the following:
   a) Unstable angina, myocardial infarction, coronary artery bypass graft surgery or coronary angioplasty within 6 months prior to the date of the Screening Visit;
   b) Transient ischemic attack or cerebrovascular accident within 6 months prior to the date of the Screening Visit;
   c) Symptomatic obstructive valvular heart disease or hypertrophic cardiomyopathy;
   d) Symptomatic congestive heart failure;
   e) Uncontrolled or recurrent ventricular tachycardia or other arrhythmia requiring an automatic implantable cardioverter defibrillator (AICD). Stable, controlled atrial fibrillation is allowed;
   f) An emergency room visit or hospitalization for confirmed cardiovascular disease within 6 months prior to the date of the Screening Visit;
13) History of a malignancy within 5 years of the date of the Screening Visit with the following exceptions:
   a) Adequately treated carcinoma in situ of the cervix;
   b) Adequately treated basal or squamous cell cancer or other localized non-melanoma skin cancer.
14) Presence of acute pancreatitis within the past 180 days prior to the date of the Screening Visit;
15) History or presence of chronic pancreatitis;
16) Presence or history of type 1 diabetes mellitus;
17) For subjects with type 2 diabetes diagnosed prior to the date of the Screening Visit OR on Screening Visit labs (defined as HbA1c≥6.5%), subjects must have no evidence of uncontrolled and potentially unstable retinopathy or maculopathy as determined by:
   a) A fundus exam performed in the 90 days prior to the date of the Screening Visit. If there has been worsening of the subject's visual function since this historical fundus exam in the opinion of the investigator, then the fundus exam must be repeated; OR
   b) A fundus exam performed between the date of the Screening Visit and Enrollment (Day −14) Pharmacological pupil-dilation is a requirement in both of the above cases unless using a digital fundus photography camera specified for non-dilated examination;
18) Personal or first degree relative(s) history of multiple endocrine neoplasia type 2 or medullary thyroid carcinoma;
19) Treatment with GLP-1 RAs in the period from 90 days prior to the date of the Screening Visit;
20) Subjects on Vitamin E regimen ≥800 IU/day must be on a stable dose (defined as no changes in prescribed dose, new Vitamin E containing medications, or discontinuation) for at least 180 days prior to the date of the Screening Visit and in the period between the date of the Screening Visit and Enrollment (Day −14);
21) Subjects on antidiabetic medications must be on a stable dose for at least 90 days prior to the date of the Screening Visit and in the period between the date of the Screening Visit and Enrollment (Day −14).

### C. Drug Products

### Semaglutide

Semaglutide solution for injection has the composition shown above in Table 1. Semaglutide solution for injection is a colorless or almost colorless liquid, free from turbidity and essentially free from particulate matter. The PDS290 pen-injector (FlexTouch®) for semaglutide is a dial-a-dose prefilled device integrated with a 3 mL cartridge filled with semaglutide 3.0 mg/mL. The pen-injector can deliver doses from 1 to 80 dose steps in increments of 1. The user can dial up and down in order to adjust a dose.

### Cilofexor and Fisocostat

Cilofexor is supplied as 100 mg and 30 mg strength tablets, as described herein. Fisocostat is supplied as round, plain-faced, film-coated white tablets containing 20 mg firsocostat, as described herein.

### D. Dosage and Administration

Subjects take semaglutide subcutaneously with a PDS290 pen-injector at approximately the same time each week. Subjects take firsocostat and/or cilofexor tablets (if applicable) at approximately the same time each day, with or without food, swallowed whole with water.

Study drug dosing and administration is as follows, based on treatment group randomization as summarized in FIG. 2:
Treatment Group A: Semaglutide administered subcutaneously with pre-filled pen-injector once weekly.
Treatment Group B: Semaglutide administered subcutaneously with pre-filled pen-injector once weekly, one firsocostat 20 mg tablet, administered orally once daily without regard to food.
Treatment Group C: Semaglutide administered subcutaneously with pre-filled pen-injector once weekly, one cilofexor 30 mg tablet, administered orally once daily without regard to food.
Treatment Group D: Semaglutide administered subcutaneously with pre-filled pen-injector once weekly, one cilofexor 100 mg tablet, administered orally once daily without regard to food.
Treatment Group E: Semaglutide administered subcutaneously with pre-filled pen-injector once weekly, one firsocostat 20 mg tablet administered orally once daily and one cilofexor 30 mg tablet administered orally once daily, both without regard to food.

After randomization, semaglutide is initiated with a starting value of 8 (0.24 mg) for the first 4 weeks, and subsequently the value is increased every 4 weeks. The semaglutide dose escalation scale is shown in Table 2.

TABLE 2
Semaglutide Dose Escalation Schedule

| | Dose | Volume | |
|---|---|---|---|
| Product | (mg) | (μL) | Duration |
| Semaglutide 3.0 mg/mL | 0.24 | 80 | Weeks 1 through 4 |
| Semaglutide 3.0 mg/mL | 0.50 | 170 | Weeks 5 through 8 |
| Semaglutide 3.0 mg/mL | 1.0 | 340 | Weeks 9 through 12 |
| Semaglutide 3.0 mg/mL | 1.7 | 570 | Weeks 13 through 16 |
| Semaglutide 3.0 mg/mL | 2.4 | 800 | Weeks 17 through 24 |

If a subject does not tolerate the planned 4-week dose-escalation regimen due to gastrointestinal adverse events or for other reasons as judged by the investigator, the subject is allowed to stay longer at the individual dose steps.

### E. Assessments

### Clinical Laboratory Analytes

In general, subjects fast (no food or drink, except water) for approximately 10 hours prior to the blood sample collection.

The following chemistry analytes are evaluated: alanine aminotransferase (ALT), aspartate aminotransferase (AST), alkaline phosphatase (ALP), bicarbonate, blood urea nitrogen (BUN), creatine kinase (CPK), calcium, chloride, creatinine, glucose, magnesium, phosphorus, potassium, sodium, total and direct bilirubin, total protein, uric acid, and gamma-glutamyl transferase (GGT).

The following hematology factors are measured: Hematocrit (Hct), hemoglobin (Hb), platelet count, red blood cell count (RBC), white blood cell count (WBC) with differential (absolute and percentage) including lymphocytes, monocytes, neutrophils, eosinophils, basophils, and mean corpuscular volume (MCV). The coagulation panel includes INR, prothrombin time (PT), and partial thromboplastin time (PTT).

Additional tests include HIV-1 (reflex to HIV-1 RNA), HBV (HBsAg), HCV (reflex to HCV RNA) serology, homeostasis model assessment of Insulin resistance (HOMA-IR, based on fasting glucose and insulin), eGFR as calculated by MDRD, HbA1c, C-peptide, calcitonin, insulin, lipid panel, genomic sample collection, ELF™ Test, and FibroTest®. Biomarker tests include, but are not limited to C-reactive protein (CRP), NMR LipoProfile®, apolipoproteins, and adiponectin.

Creatinine Clearance eGFR

Estimated glomerular filtration rate (eGFR) is determined by creatinine clearance, which is calculated by the Modification of Diet in Renal Disease (MDRD) Study equation:

eGFR(mL/min/1.73 m²)=175×Serum Creatinine^{-1.154}×(Age)^{-0.203}×(1.212 if African American)×(0.742 if female).

Serum creatinine in μmol/L is rounded to zero decimal places and converted to mg/dL by multiplying by 0.01131 prior to applying the formula. Creatinine in mg/dL is rounded to 2 decimal places prior to applying formula.

### De Novo Lipogenesis (DNL)

To analyze the pharmacodynamic effects of the study drugs, subjects drink 45 mL of deuterated water three times per day starting on Day 154 through Day 160. Kinetic biomarker samples are obtained on Day 154, Day 157, Day 161 and Day 168. The assessment involves the analysis of DNL values; specifically, the change (absolute and relative) from baseline between the post-dose and pre-dose deuterated water loading periods.

### Child-Pugh (CP) Score

The Child-Pugh (CP) score is used to assess the prognosis of chronic liver disease, primarily cirrhosis.

### MRE and MRI-PDFF

Liver stiffness is assessed by MRE (shear wave 60 Hz) and MRI-PDFF at the Screening Visit, Day 84 (Week 12) and Day 168 (Week 24).

### FibroScan®

FibroScan® examinations are performed at the Screening Visit, Day 84 (Week 12) and Day 168 (Week 24) and median liver stiffness in kilopascals (kPa), interquartile range/median value (IQR/M), and success rate (number of valid shots/total number of shots) are assessed. Where available, the median CAP and the interquartile range of CAP values are recorded from FibroScan® examinations.

### Additional Screening

Standard 12-lead electrocardiogram (ECG) assessments are performed at Screening and the Day 168 (Week 24) visit. The Investigator reviews the ECGs for any clinically significant abnormalities to ensure subject safety.

For subjects with type 2 diabetes (from medical history or from Screening Hemoglobin A1c≥6.5%), a fundus exam is performed at Screening. Fundus examinations require pharmacological dilation of both pupils or the use of a digital fundus photography camera specified for non-dilated examination.

Health-related quality of life is also assessed. The Chronic Liver Disease Questionnaire-Nonalcoholic Fatty Liver Disease (CLDQ-NAFLD) asks questions related to liver disease and specifically NAFLD, to measure health related quality of life in subjects with chronic liver disease. The EuroQol Five Dimensions (EQ-5D) questionnaire not disease specific, but is a standard measure of health status developed by the EuroQol Group to provide a simple, generic measure of health for clinical and economical appraisal. The tool consists of the EQ-5D descriptive system and the EQ Visual Analog Scale (VAS). The descriptive part comprises 5 dimensions (mobility, self-care, usual activities, pain/discomfort, and anxiety/depression). Each of these 5 dimensions has 5 levels (no problem, slight problems, moderate problems, severe problems and unable to). Results for each of the 5 dimensions are combined into a 5-digit number to describe the subject's health state. The VAS records the subject's health on a 0-100 mm VAS scale, with 0 indicating “the worst health you can imagine” and 100 indicating “the best health you can imagine”.

### F. Endpoints

The primary endpoint of this study is the safety and tolerability of study drugs in subjects with NASH.

The exploratory endpoints of this study include non-invasive measures of fibrosis and steatosis, including:
change from baseline in steatosis as measured by MRI-PDFF;
change from baseline in steatosis as measured by CAP;
change from baseline in liver stiffness as measured by MRE;
change from baseline in liver stiffness as measured by FibroScan®;
change from baseline in kinetic parameters of markers of de novo lipogenesis (DNL), fibrogenesis, and inflammation;
change from baseline in markers of liver injury and function, including ALT, AST, bilirubin, GGT, and ALP;
change from baseline in non-invasive markers of fibrosis, including ELF™ Test score and FibroTest®;
change from baseline in HOMA-IR, HbA1c, serum lipid profiles, and systolic and diastolic blood pressure;
change from baseline in body weight; and
change from baseline in health related quality of life endpoints based on, for example, CLDQ-NAFLD and EQ-5D.

The baseline for the endpoints above is prior to the first treatment of the study.

Kinetic biomarkers are analyzed to evaluate the pharmacodynamic effects of study drugs. The assessment involves the analysis of de novo lipogenesis (DNL) values; specifically, the change (absolute and relative) from baseline between the post-dose and pre-dose deuterated water loading periods. For other biomarkers, the exploratory analyses are performed by providing descriptive statistics of biomarker expression and change from baseline at each sampling time by treatment. Point estimates and 95% confidence intervals may be calculated. Exploratory analyses may also be performed to evaluate the association of individual exploratory biomarkers or combination of biomarkers with clinical measurements and other risk factors.

### G. Data and Results

### Participants

Patients aged 18-75 years were eligible for inclusion if they had a historical liver biopsy within 6 months of screening consistent with NASH (defined as the presence of steatosis, inflammation, and ballooning) with stage 2 or 3 fibrosis according to the NASH Clinical Research Network (CRN) classification, or if they met all four criteria of: a clinical diagnosis of NAFLD, magnetic resonance imaging proton density fat fraction (MRI-PDFF) with ≥10% steatosis, liver stiffness by transient elastography (TE; FibroScan®, Echosens, Paris, France)≥7 kPa (consistent with fibrosis), and FibroTest®<0.75 (to exclude cirrhosis). Other inclusion criteria included alanine aminotransferase (ALT)≤5×upper limit of normal, HbA_{1c}≤9.5%, body weight >60 kg, and body mass index (BMI)>23 kg/m². Key exclusion criteria included a historical liver biopsy consistent with cirrhosis, history of decompensated liver disease, liver transplantation or hepatocellular carcinoma, other causes of liver disease, excessive alcohol consumption (>21 oz/week for men or 14 oz/week for women), unstable cardiovascular disease, or weight loss >5% within 6 months prior to screening. Certain eligibility criteria are listed in the Inclusion Criteria and Exclusion Criteria sections above. All participants provided written, informed consent before any trial-related activities.

### Randomization and Masking

Patients were randomized (1:1:1:1:1) to one of five treatment groups:
semaglutide monotherapy, semaglutide plus cilofexor 30 mg (SEMA+CILO 30), semaglutide plus cilofexor 100 mg (SEMA+CILO 100), semaglutide plus firsocostat 20 mg (SEMA+FIR), or semaglutide plus cilofexor 30 mg plus firsocostat 20 mg (SEMA+CILO+FIR). An interactive mobile/web response system (IXRS) was used for centralized randomization and treatment assignment. Randomization was stratified by the presence or absence of type 2 diabetes. Treatment was open-label.

### Procedures

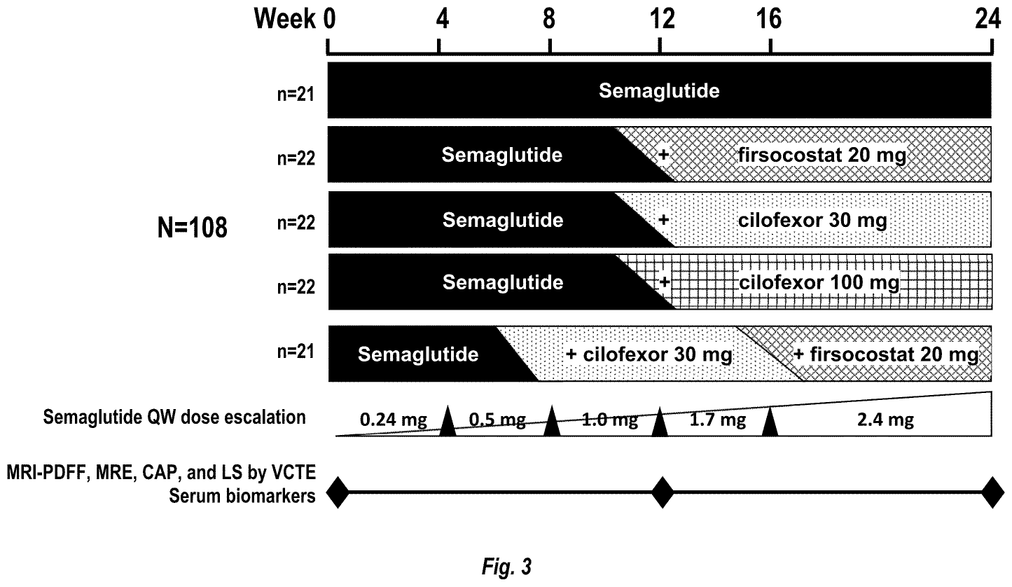

The trial consisted of a 2-week screening period, a 2-week pre-treatment period, a 24-week treatment period, and a 7-week follow-up period (FIG. 3).

Semaglutide was administered by subcutaneous injection once weekly with a prefilled pen-injector and was initiated at a starting dose of 0.24 mg, which was increased at 4-week intervals (to 0.5 mg, 1.0 mg, and 1.7 mg) until the recommended target dose of 2.4 mg was reached (from week 17 onwards). Patients who could not tolerate the planned dose-escalation schedule were encouraged to extend any single dose step for a maximum of one additional week, and to re-attempt dose escalation to 2.4 mg at least once. Cilofexor and firsocostat were given orally once daily with or without food.

Imaging assessments (MRI-PDFF, liver stiffness by 2-D magnetic resonance elastography [MRE] and TE) were conducted at screening (or within 4 weeks prior to screening), and at weeks 12 and 24. TE was performed by experienced operators, and MRI-PDFF and MRE images were analyzed by a central reader as previously described [Patel et al. Hepatology. 2020; 72(1):58-71; Loomba et al. Gastroenterology. 2018; 155(5):1463-1473; Loomba et al. Hepatology. 2020 Nov. 10. doi: 10.1002/hep.31622]. Serum samples were collected at screening, baseline, and every 4 weeks through to week 24 for clinical laboratory values; blood biomarkers were assessed at screening, baseline, and weeks 12 and 24.

### Outcomes

Safety assessments included adverse events, clinical laboratory assessments, vital signs, electrocardiograms (ECG), and physical examination. Clinical and laboratory adverse events were coded using the Medical Dictionary for Regulatory Activities (MedDRA), version 23.0.

All efficacy endpoints were exploratory and included changes from baseline at week 24 in liver steatosis as measured by MRI-PDFF and controlled attenuation parameter (CAP; FibroScan®). Proportions of patients with ≥5% absolute and ≥30% relative reductions in MRI-PDFF at week 24 were also assessed. In addition to these protocol-defined analyses, post-hoc assessments included proportions of patients with ≥50% relative reduction in MRI-PDFF and MRI-PDFF normalization (defined as <5%) at week 24. Changes in liver biochemistry (ALT, aspartate aminotransferase [AST], alkaline phosphatase [ALP], gamma-glutamyl transferase [GGT], total and direct bilirubin), platelets, albumin, international normalized ratio (INR), Model for End-Stage Liver Disease (MELD) score, NITs of fibrosis including Enhanced Liver Fibrosis (ELF) test score (and proportion of patients with a ≥0.5-unit reduction), FibroSure/FibroTest score, and markers of inflammation and apoptosis (C-reactive protein [CRP], CK-18 M30) were evaluated. Changes in liver stiffness were assessed by TE and MRE, including the proportion of patients with a ≥25% relative reduction in liver stiffness by TE. Changes in FibroScan-AST (FAST) score, which combines liver stiffness measured by TE, steatosis by CAP, and serum AST for the non-invasive identification of patients with NASH and ≥F2 fibrosis, were also assessed post-hoc [Newsome Lancet Gastroenterol Hepatol. 2020 April; 5(4):362-373]. Finally, metabolic parameters (body weight, BMI, HbA_{1c}, fasting plasma glucose, fasting insulin, homeostatic model assessment of insulin resistance [HOMA-IR]), blood pressure, estimated glomerular filtration rate (eGFR), and serum creatinine were assessed.

### Statistical Analyses

Due to the exploratory nature of this trial, sample size was based on clinical experience with prior studies rather than a formal power calculation.

Safety analyses were performed on the safety analysis set, which included all patients who took at least one dose of any study drug. Efficacy analyses were performed on the full analysis set (FAS), which included all randomized patients who received at least one dose of any study drug. Since all dosed subjects were randomized, these two analysis sets included the same patients.

All protocol-defined, efficacy analyses were exploratory in nature and descriptive statistics are provided. Comparative analyses between combination regimens and semaglutide monotherapy were conducted post-hoc. For evaluation of changes from baseline to week 24, analysis of covariance (ANCOVA) with adjustment for baseline value and diabetes status was used, and least-squares (LS) means, LS mean differences, associated 95% confidence intervals (CI), and p-values were calculated. Proportions of binary responders were compared by Fisher's exact test.

Sensitivity analyses were conducted for these parameters excluding patients in whom imaging data was collected more than 30 days after the last dose of study drug(s).

Nominal p-values are reported. All comparisons were made at a significance level of 0.05. All analyses were based on observed data and were done using SAS version 9.4 (SAS; Cary, N.C.).

### Results

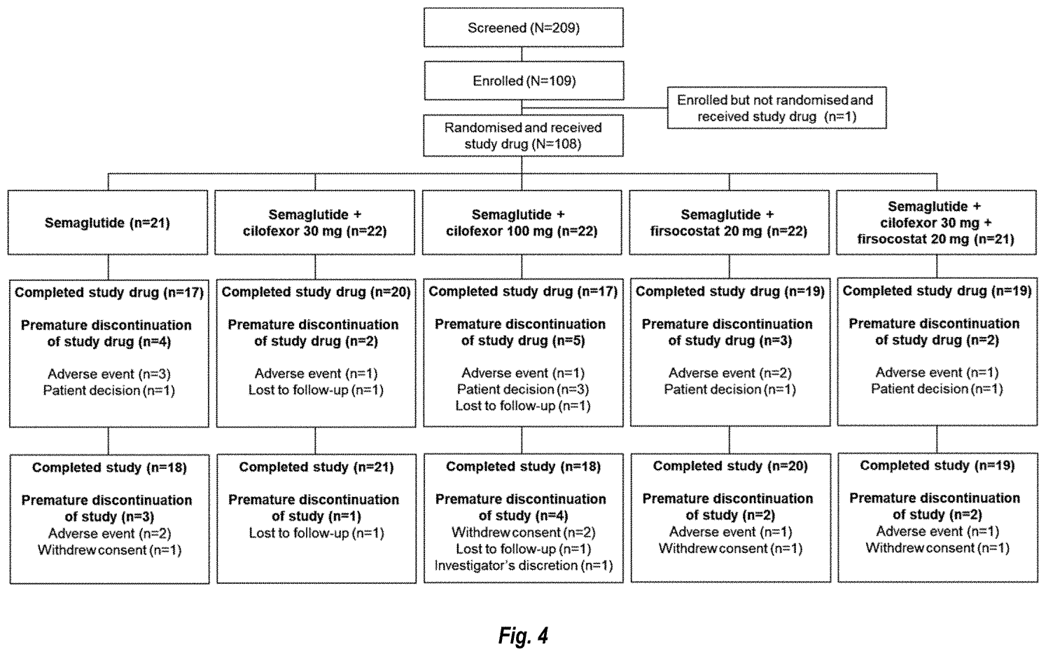

A total of 209 patients were screened and 108 of those patients were randomized to 24 weeks of treatment with SEMA (semaglutide) (n=21), SEMA+CILO 30 (n=22), SEMA+CILO 100 (n=22), SEMA+FIR (n=22), or SEMA+FIR+CILO 30 (n=21). All 108 patients who were randomized received at least one dose of any study drug. Ninety-two patients (85%) completed study drug treatment and 96 (89%) completed the study (FIG. 4). The majority of patients in each group had treatment adherence rates of 90-100%. Overall, 89 patients (82%) achieved the semaglutide target dose of 2.4 mg with no meaningful differences between semaglutide alone or combination treatments or across the combination groups.

Across treatment groups, most patients were female (68.5%), white (85.2%), and had type 2 diabetes (54.6%); the median (Q1-Q3) age was 54 years (48-61). At baseline, NITs were consistent with mild-to-moderate fibrosis (median ELF 9.4 [8.9, 9.9]; and liver stiffness by TE 9.3 kPa [7.7, 12.0]) and moderate-to-severe steatosis (median MRI-PDFF 17.9% [12.0, 24.3]). Baseline demographic and clinical characteristics were similar between the groups (Table 3), although median body weight and liver stiffness by TE were higher, and serum ALT and AST were lower in the combination groups compared with the semaglutide group.

TABLE 3
Baseline demographics and clinical characteristics

| | | | | |
|---|---|---|---|---|
| | | | | Semaglutide 2.4 mg + |
| Semaglutide | Semaglutide 2.4 mg + | Semaglutide 2.4 mg + | Semaglutide 2.4 mg + | cilofexor 30 mg + |
| 2.4 mg | cilofexor 30 mg | cilofexor 100 mg | firsocostat 20 mg | firsocostat 20 mg |
| (n = 21) | (n = 22) | (n = 22) | (n = 22) | (n = 21) |

Demographics

| | | | | | | | | | | |
|---|---|---|---|---|---|---|---|---|---|---|
| Age, y | 57 | (51, 61) | 53 | (46, 58) | 58 | (46, 61) | 53 | (47, 60) | 53 | (49, 61) |
| Female sex, n (%) | 15 | (71) | 16 | (73) | 13 | (59) | 15 | (68) | 15 | (71) |
| Race, white, n (%) | 17 | (81) | 18 | (82) | 19 | (86) | 19 | (86) | 19 | (90) |
| Ethnicity, Hispanic, n | 8 | (38) | 5 | (23) | 7 | (32) | 12 | (55) | 11 | (52) |

(%)
Metabolic parameters

| | | | | | | | | | | |
|---|---|---|---|---|---|---|---|---|---|---|
| Body weight, kg | 91.4 | (77.2, 104.8) | 87.9 | (80.7, 115.3) | 98.4 | (83.1, 114.3) | 91.3 | (79.7, 112.7) | 113.0 | (92.9, 116.2) |
| Body mass index, kg/m² | 33.2 | (30.9, 36.4) | 34.0 | (32.7, 36.0) | 34.1 | (29.7, 39.4) | 33.5 | (30.2, 37.9) | 39.5 | (34.3, 43.0) |
| Diabetes, n (%) | 11 | (52) | 12 | (55) | 12 | (55) | 13 | (59) | 11 | (52) |
| Fasting plasma glucose, | 104 | (98, 120) | 110 | (98, 132) | 117 | (94, 153) | 111 | (92, 136) | 118 | (100, 148) |
| mg/dL | | | | | | | | | | |
| HbA_{1c}, % | 6.1 | (5.8, 6.7) | 6.3 | (5.7, 7.7) | 6.5 | (5.6, 7.8) | 6.3 | (5.9, 7.3) | 6.3 | (5.9, 7.5) |
| Total cholesterol, mg/dL | 188 | (159, 219) | 168 | (157, 205) | 170 | (152, 190) | 169 | (154, 190) | 178 | (151, 211) |
| LDL cholesterol, mg/dL | 101 | (85, 129) | 91 | (72, 112) | 87 | (71, 111) | 103 | (71, 113) | 107 | (81, 134) |
| HDL cholesterol, mg/dL | 44 | (37, 53) | 40 | (36, 51) | 42 | (37, 49) | 43 | (40, 48) | 40 | (36, 44) |
| VLDL cholesterol, | 33 | (21, 42) | 35 | (23, 46) | 36 | (27, 45) | 32 | (23, 42) | 31 | (26, 39) |
| mg/dL | | | | | | | | | | |
| Triglycerides, mg/dL | 167 | (104, 230) | 179 | (114, 260) | 177 | (137, 225) | 160 | (116, 212) | 155 | (131, 197) |

Markers of liver injury and function

| | | | | | | | | | | |
|---|---|---|---|---|---|---|---|---|---|---|
| ALT, U/L | 60 | (48, 98) | 53 | (40, 86) | 40 | (24, 74) | 45 | (29, 76) | 42 | (35, 70) |
| AST, U/L | 50 | (36, 61) | 41 | (31, 57) | 27 | (22, 53) | 43 | (26, 51) | 34 | (27, 44) |
| ALP, U/L | 78 | (62, 92) | 79 | (63, 94) | 82 | (68, 103) | 82 | (64, 105) | 78 | (62, 97) |
| GGT U/L | 35 | (30, 56) | 71 | (32, 105) | 33 | (25, 81) | 38 | (26, 94) | 33 | (22, 48) |
| Total bilirubin, mg/dL | 0.5 | (0.4, 0.7) | 0.5 | (0.4, 0.7) | 0.5 | (0.3, 0.7) | 0.4 | (0.4, 0.6) | 0.4 | (0.3, 0.5) |
| Platelets, ×10³/μL | 258 | (214, 322) | 254 | (235, 293) | 274 | (238, 338) | 265 | (228, 314) | 262 | (218, 311) |

Non-invasive markers of steatosis and fibrosis

| | | | | | | | | | | |
|---|---|---|---|---|---|---|---|---|---|---|
| MRI-PDFF, % | 15.3 | (9.9, 19.5) | 19.3 | (13.5, 27.1) | 18.0 | (14.5, 23.9) | 18.1 | (12.5, 24.3) | 15.9 | (12.3, 25.4) |
| CAP by TE, dB/m | 319 | (294, 364) | 347 | (319, 370) | 359 | (323, 389) | 350 | (325, 374) | 336 | (324, 376) |
| Liver stiffness by TE, | 7.7 | (7.2, 10.0) | 8.8 | (7.6, 14.1) | 10.5 | (7.6, 12.5) | 9.4 | (8.1, 11.3) | 10.7 | (8.8, 12.3) |
| kPa | | | | | | | | | | |
| Liver stiffness by MRE, | 2.8 | (2.4, 3.3) | 3.0 | (2.3, 3.8) | 2.5 | (2.4, 3.0) | 3.0 | (2.6, 3.8) | 2.9 | (2.6, 3.6) |
| kPa | | | | | | | | | | |
| ELF score | 9.6 | (9.0, 10.1) | 9.4 | (8.8, 10.1) | 9.4 | (8.9, 9.7) | 9.4 | (8.6, 9.9) | 9.4 | (8.9, 9.9) |
| FAST score | 0.56 | (0.50, 0.69) | 0.61 | (0.42, 0.71) | 0.47 | (0.24, 0.76) | 0.56 | (0.33, 0.69) | 0.53 | (0.39, 0.66) |

Continuous pammeters are median (Q1, Q3).
ALT, alanine aminotransferase;
ALP, alkaline phosphatase;
AST, aspartate aminotransferase;
BMI, body mass index;
CAP, controlled attenuation parameter;
eGFR, estimated glomerular filtration rate;
ELF, Enhanced Liver Fibrosis;
FAST, FibroScan-AST;
GGT, gamma-glutamyl transferase;
HbA_{1c}, haemoglobin A_{1c};
HDL, high-density lipoprotein;
HOMA-IR, homeostasis mode assessment of insulin resistance;
LDL, low-density lipoprotein;
MRE, magnetic resonance elastography;
MRI-PDFF, magnetic resonance imaging-proton density fat fraction;
TE, transient elastography;
VLDL, very low-density lipoprotein.

TABLE 4
Adverse events, laboratory abnormalities and changes in serum lipids

| | | | | |
|---|---|---|---|---|
| | | | | Semaglutide 2.4 mg + |
| Semaglutide | Semaglutide 2.4 mg + | Semaglutide 2.4 mg + | Semaglutide 2.4 mg + | cilofexor 30 mg + |
| 2.4 mg | cilofexor 30 mg | cilofexor 100 mg | firsocostat 20 mg | firsocostat 20 mg |
| (n = 21) | (n = 22) | (n = 22) | (n = 22) | (n = 21) |

Safety overview

| | | | | | | | | | | |
|---|---|---|---|---|---|---|---|---|---|---|
| Any adverse event | 17 | (81) | 18 | (82) | 16 | (73) | 19 | (86) | 19 | (90) |
| Grade ≥ 2 adverse event | 10 | (48) | 9 | (41) | 9 | (41) | 10 | (45) | 10 | (48) |
| Treatment-related adverse event | 13 | (62) | 17 | (77) | 11 | (50) | 15 | (68) | 15 | (71) |
| Treatment-related grade ≥ 2 | 4 | (19) | 3 | (14) | 7 | (32) | 7 | (32) | 5 | (24) |
| adverse event | | | | | | | | | | |
| Serious adverse event | 1 | (5) | 0 | | 1 | (5) | 0 | | 0 | |
| Adverse event leading to | 3 | (14) | 1 | (5) | 1 | (5) | 2 | (9) | 1 | (5) |
| discontinuation | | | | | | | | | | |
| of any study drug | | | | | | | | | | |
| Death | 0 | | 0 | | 0 | | 0 | | 0 | |

Most common adverse events (>10% of subjects in any treatment group)

| | | | | | | | | | | |
|---|---|---|---|---|---|---|---|---|---|---|
| Nausea | 9 | (43) | 8 | (36) | 6 | (27) | 3 | (14) | 14 | (67) |
| Diarrhea | 5 | (24) | 5 | (23) | 1 | (5) | 4 | (18) | 3 | (14) |
| Constipation | 2 | (10) | 5 | (23) | 3 | (14) | 2 | (9) | 5 | (24) |
| Decreased appetite | 4 | (19) | 6 | (27) | 0 | | 4 | (18) | 3 | (14) |
| Vomiting | 2 | (10) | 2 | (9) | 1 | (5) | 2 | (9) | 6 | (29) |
| Abdominal pain | 1 | (5) | 1 | (5) | 1 | (5) | 4 | (18) | 2 | (10) |
| Fatigue | 2 | (10) | 3 | (14) | 1 | (5) | 1 | (5) | 2 | (10) |
| Dizziness | 2 | (10) | 3 | (14) | 2 | (9) | 1 | (5) | 0 | |
| Abdominal pain upper | 3 | (14) | 1 | (5) | 0 | | 1 | (5) | 2 | (10) |
| Arthralgia | 5 | (24) | 0 | | 2 | (9) | 0 | | 0 | |
| Headache | 1 | (5) | 1 | (5) | 1 | (5) | 3 | (14) | 1 | (5) |
| Hypoglycaemia | 0 | | 4 | (18)* | 0 | | 2 | (9) | 0 | |
| Gastroenteritis | 0 | | 0 | | 3 | (14) | 1 | (5) | 1 | (5) |
| Gastroesophageal reflux disease | 0 | | 1 | (5) | 0 | | 4 | (18) | 0 | |
| Early satiety | 1 | (5) | 0 | | 0 | | 3 | (14) | 0 | |
| Abdominal distension | 0 | | 0 | | 0 | | 3 | (14) | 0 | |

Adverse event of interest

| | | | | | | | | |
|---|---|---|---|---|---|---|---|---|
| Pruritus | 0 | 1 | (5) | 2 | (9) | 0 | 2 | (10) |

Laboratory abnormalities

| | | | | | | | |
|---|---|---|---|---|---|---|---|
| Grade 3 | 0 | 0 | 0 | | l | (5)^{†} | 0 |
| Grade 4 | 0 | 0 | 2 | (9)^{‡} | 0 | | 0 |

Lipid changes, least squares mean change from baseline to week 24 (95% CI)

| | | | | | | | | | | |
|---|---|---|---|---|---|---|---|---|---|---|
| Total cholesterol, mg/dL | −15 | (−29, −1) | −9 | (−21, 3) | 15 | (2, 29)^{§} | −7 | (−20, 6) | 3 | (−10, 16) |
| LDL cholesterol, mg/dL | −9 | (−22, 4) | 0 | (−11, 12) | 23 | (11, 36)^{§} | −5 | (−17, 7) | 7 | (−6, 21) |
| HDL cholesterol, mg/dL | −1 | (−3, 2) | −2 | (−4, 0) | −4 | (−7, −1) | −5 | (−8, −03)^{§} | −5 | (−8, −2)^{§} |
| VLDLcholesterol, mg/dL | −7 | (−12, −2) | −5 | (−9, −1) | −5 | (−9, 0) | 4 | (0, 8)S | 1 | (−4, 5)^{§} |
| Triglycerides, mg/dL | −28 | (−60, 4) | −43 | (−72, −14) | −16 | (−47, 15) | 15 | (−16, 46) | 26 | (−7, 58)^{§} |

Data are n (%) except for lipid changes.
*One patient was reported to experience hypoglycaemia but not a hypoglycaemic episode (defined as plasma glucose levels of ≤70 mg/dL).
^{†}Grade 3 hypertriglyceridaemia at week 4 (577 mg/dL) in a patient with grade 2 elevation at baseline (487 mg/dL).
^{‡}Grade 4 creatine phosphokinase elevations in 2 patients, neither attributed to study drug.
^{§}p < 0.05 vs semaglutide alone.
CI, confidence interval;
HDL, high-density lipoprotein;
LDL, low-density lipoprotein;
VLDL, very low-density lipoprotein.

### Safety Assessments

The majority of patients experienced at least one adverse event, with similar rates observed across treatment groups (73% to 90%; Table 4). Most adverse events were grade 1 or 2 in severity; rates of grade ≥2 events were also similar across groups (41% to 48%). The most commonly reported adverse events were gastrointestinal, including nausea, diarrhea, constipation, and decreased appetite. A higher rate of nausea was observed in the semaglutide+firsocostat+cilofexor group versus semaglutide alone; however, this did not result in increased treatment discontinuations. Pruritus was observed in only five patients, all on cilofexor (one [4.5%] in the semaglutide+cilofexor 30 group, two [9.1%] in the semaglutide+cilofexor 100 group, and two [9.5%] in the semaglutide+firsocostat+cilofexor group). All pruritus events were mild and none led to treatment discontinuation. Ten hypoglycaemic episodes were reported in five patients (4.6%); all were grade 1 or 2 in severity and dose modification of semaglutide was required in only one patient.

Only two patients had serious adverse events, one in the semaglutide group (grade 3 diarrhea and vomiting) and one in the semaglutide+cilofexor 100 group (grade 3 pancreatitis); in both patients, study drug was discontinued. Overall, eight patients (7.4%) discontinued any study drug and 15 patients (13.9%) required dose modification or interruption of any study drug due to adverse events, mostly gastrointestinal in nature. Drug discontinuation due to adverse events was not increased in the combination groups versus semaglutide monotherapy. No deaths occurred during the trial.

Changes in serum lipids between baseline and week 24 are summarized in Table 4. Semaglutide resulted in improvements in triglycerides, and total, LDL, and VLDL cholesterol. While LDL cholesterol increased at week 24 in the semaglutide+cilofexor 100 group, no change was observed in patients treated with cilofexor 30 mg. Increases in triglycerides were observed in firsocostat-containing groups, including one patient with grade 3 hypertriglyceridaemia in the semaglutide+firsocostat group. In this patient, triglycerides increased from 487 mg/dL (grade 2) at baseline to 577 mg/dL (grade 3) at week 4, after which the patient remained on study drug with no further grade 3 or 4 triglyceride elevations. Otherwise, grade ≥3 laboratory abnormalities were reported in two patients in the semaglutide+cilofexor 100 group; both were grade 4 increases in blood creatine phosphokinase considered unrelated to study treatment. No evidence of drug-related hepatotoxicity was observed. At week 24, median increases in heart rate of 1 to 10 bpm were observed across treatment groups, with no abnormal or clinically significant ECG findings or other clinically relevant changes in vital signs.

Exploratory Efficacy Endpoints Hepatic steatosis by MRI-PDFF and CAP

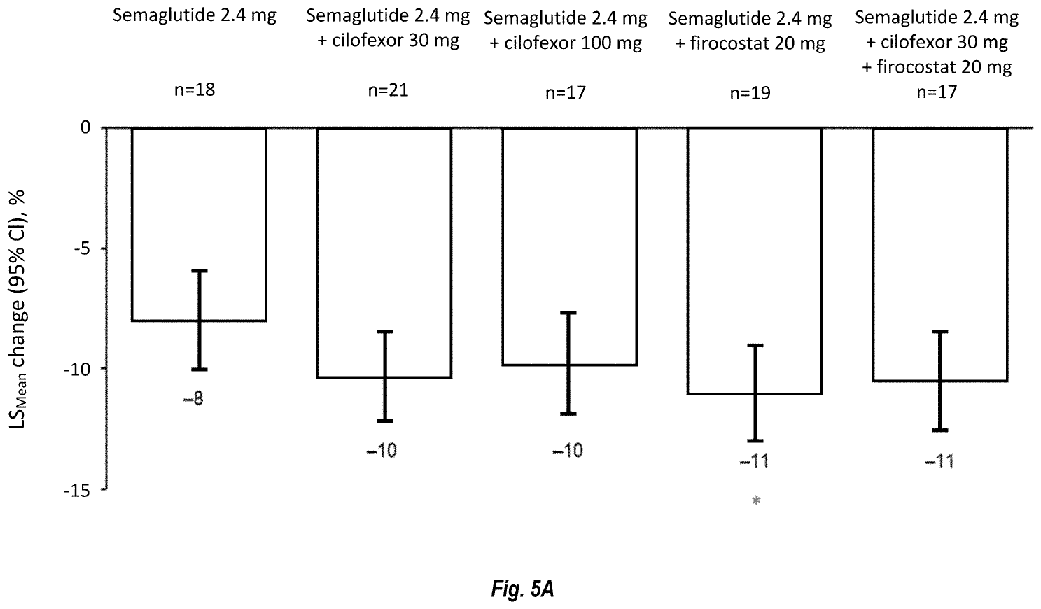

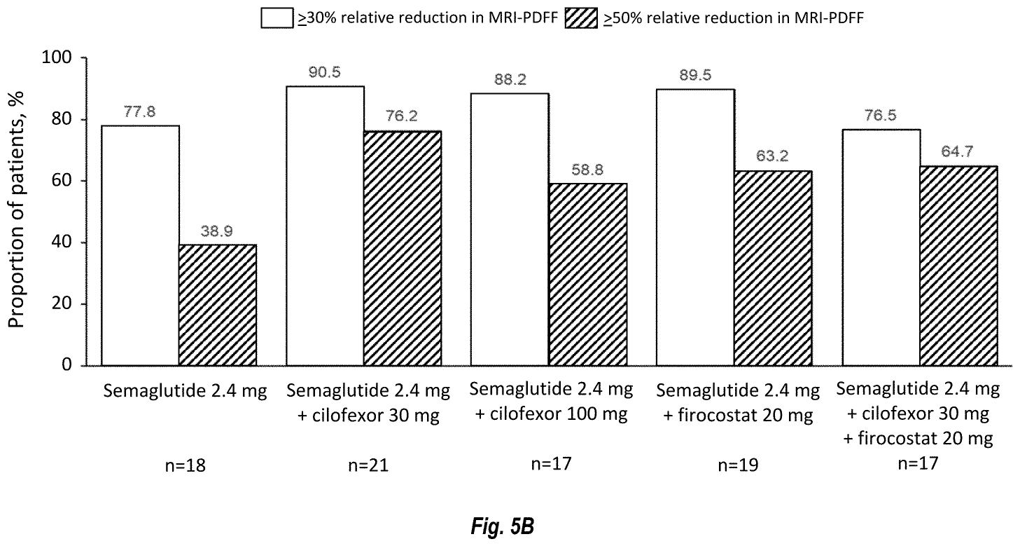

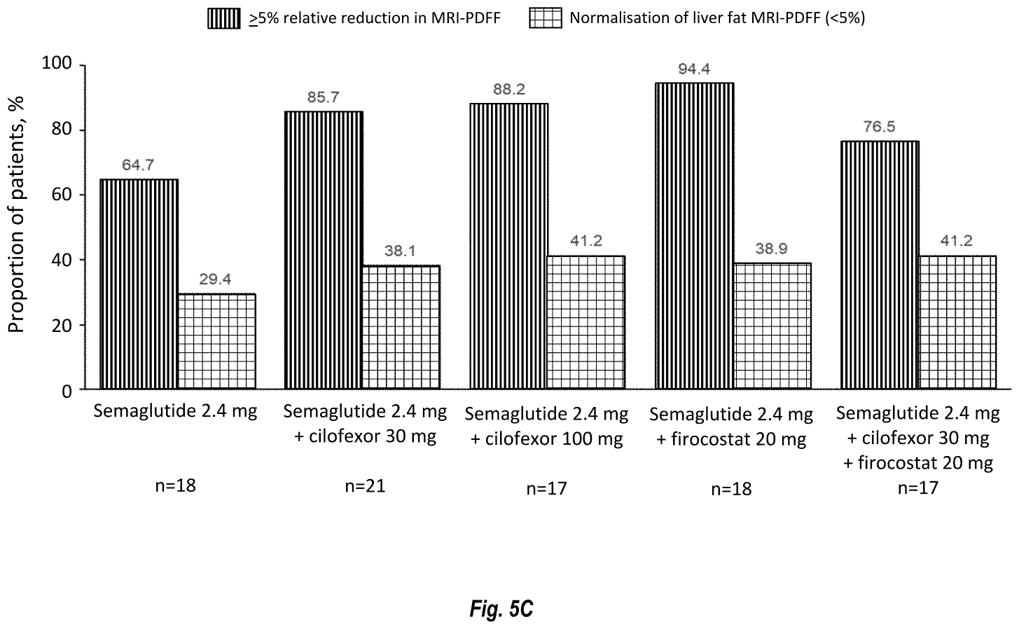

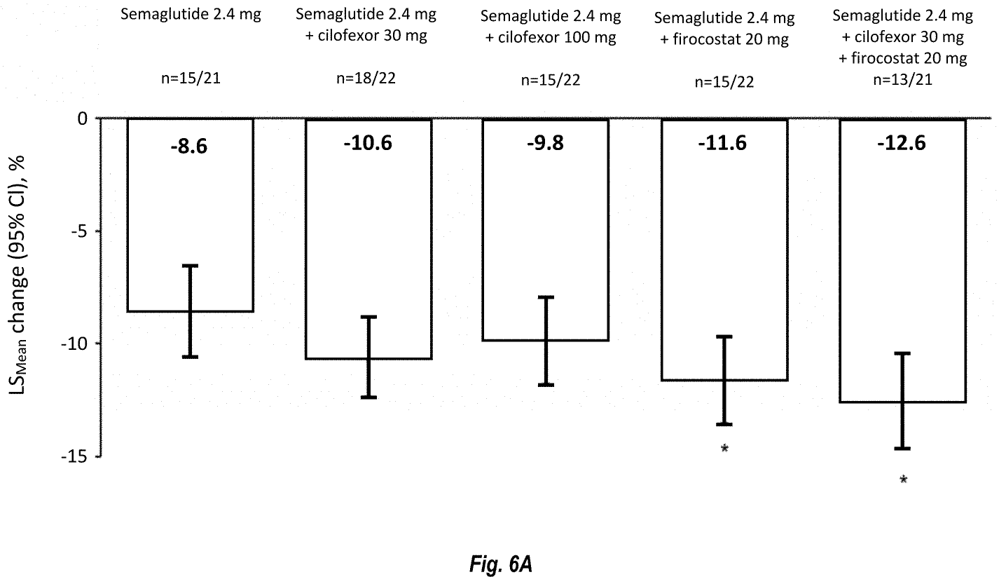

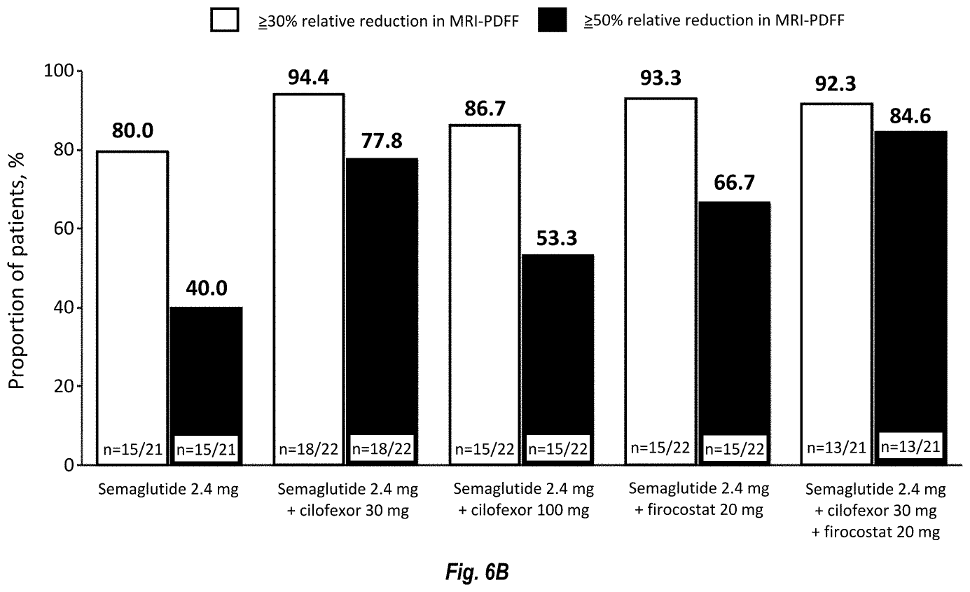

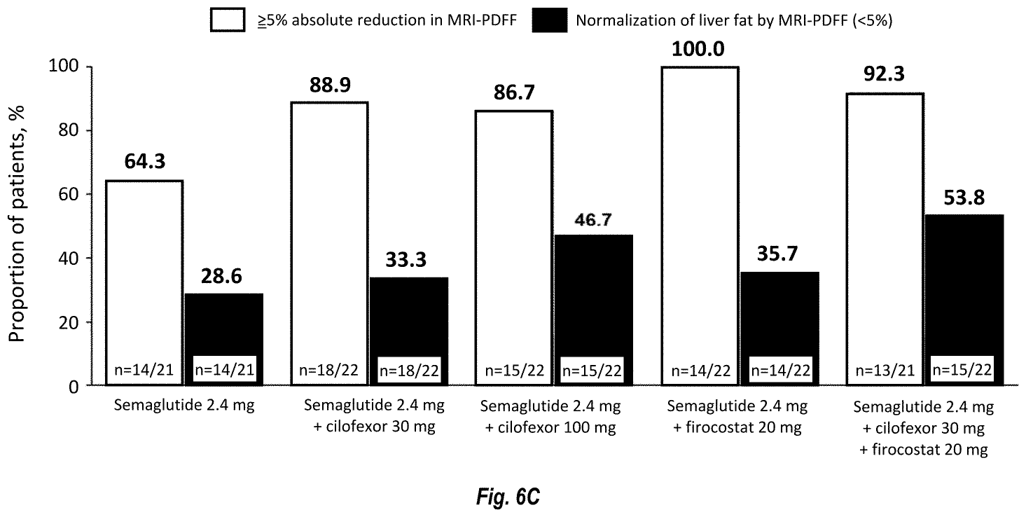

Changes from baseline to week 24 in hepatic steatosis as measured by MRI-PDFF are shown in FIG. 5A. Compared with the semaglutide monotherapy group (LS mean change: −8.0%), greater absolute reductions were observed in the combination treatment groups (LS mean change range: −9.8% to −11.0%). This difference was statistically significant between the semaglutide and semaglutide+firsocostat groups (p=0.0353). Similar results were observed in a sensitivity analysis that excluded patients with imaging data collected at least 30 days after the last dose of study drug, although the difference between semaglutide and the semaglutide+firsocostat+cilofexor group (LS mean change: −8.6% versus −12.6%) was also significant (p=0.0078) in this analysis (FIG. 6A-C).

Greater median relative reductions in MRI-PDFF at Week 24 were observed in the combination groups (−55.7 to −59.4%) versus with semaglutide monotherapy (−46.2%). More patients in the combination groups achieved relative reductions in MRI-PDFF of >30% and ≥50% compared with the semaglutide group (FIG. 5B). A ≥5% absolute reduction in liver fat was achieved by 64.7% of patients in the semaglutide group versus 76.5% to 94.4% in the combination groups (FIG. 5C). Normalization of liver fat by MRI-PDFF to ≤5% in patients with ≥5% liver fat at baseline was achieved by 38.1% to 41.2% of patients treated with combinations versus 29.4% with semaglutide (FIG. 5C).

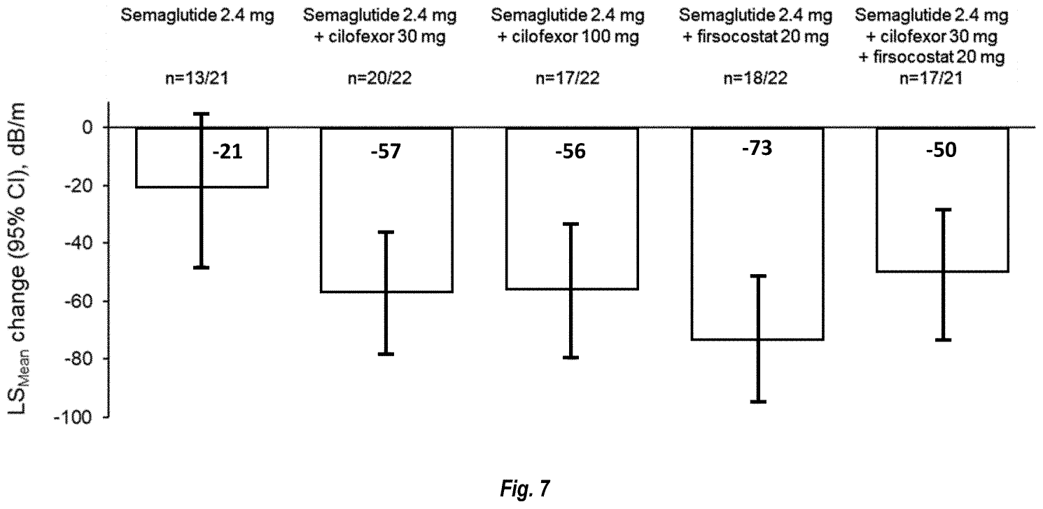

Improvements in hepatic steatosis were also observed when assessed by CAP, with median reductions of 52 to 80 dB/m for the combination groups compared with 21 dB/m with semaglutide (FIG. 7). These reductions were statistically significant for the semaglutide+firsocostat (p=0.0034) and semaglutide+cilofexor 30 (p=0.0379) groups versus semaglutide.

### Liver Biochemistry

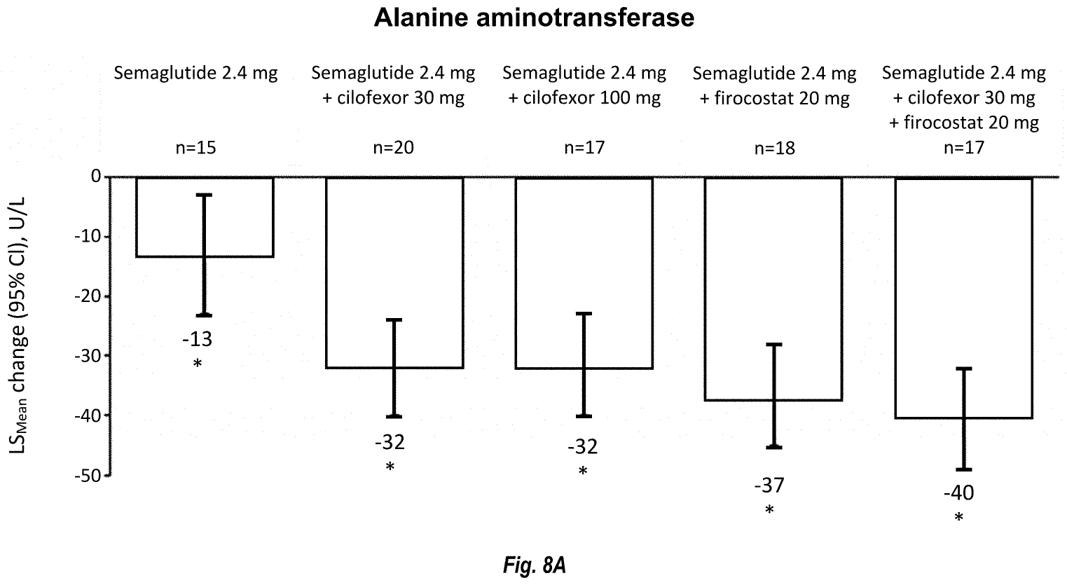

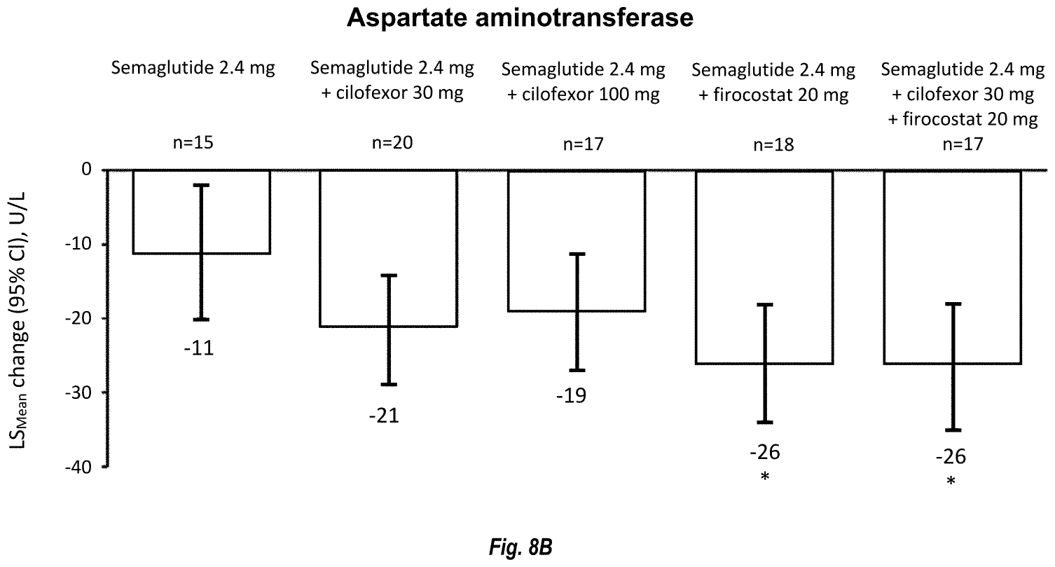

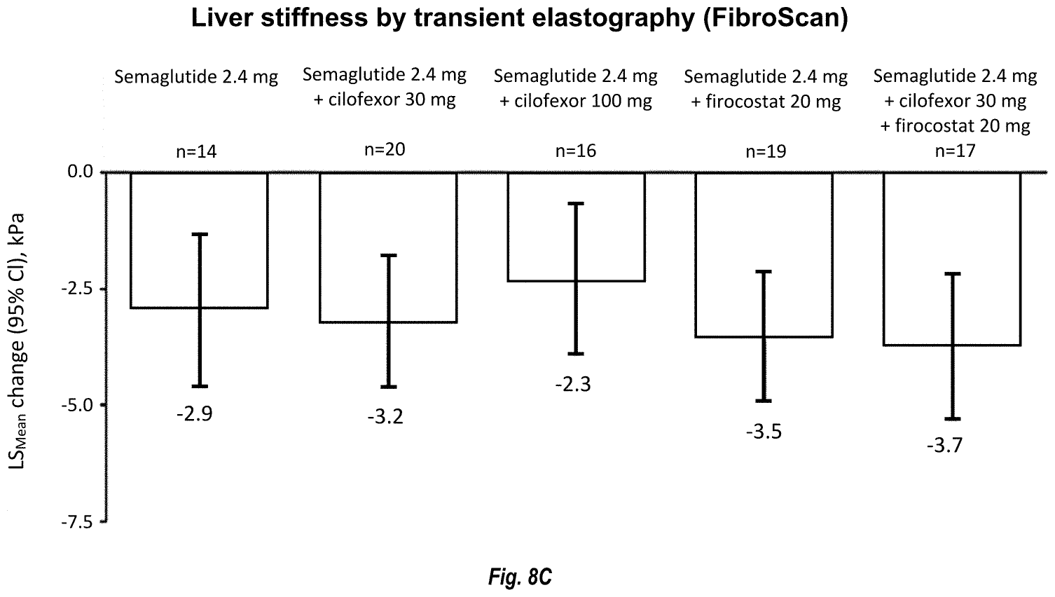

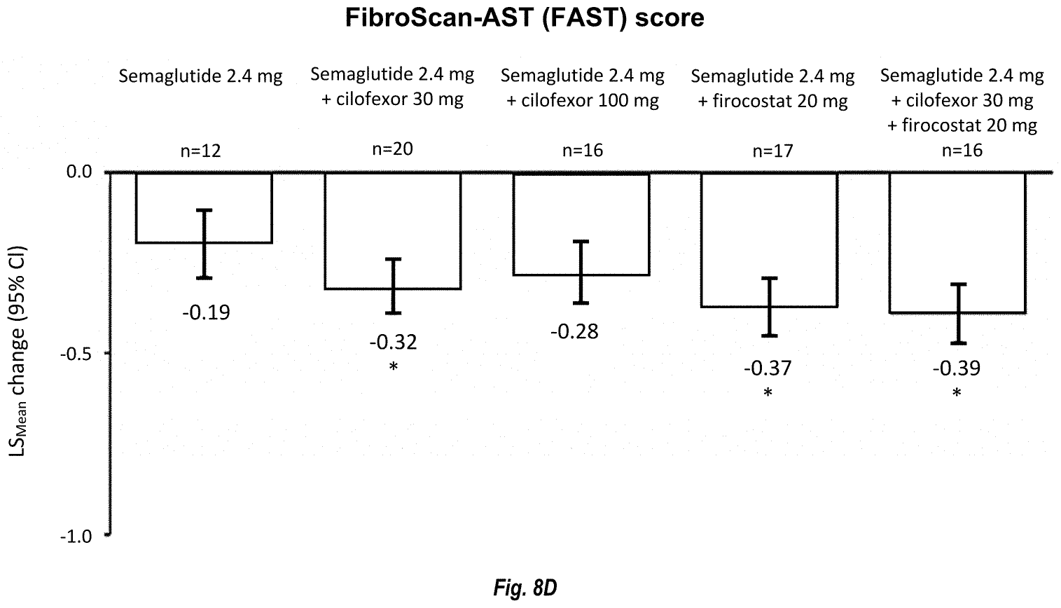

Across treatment groups, reductions from baseline in serum ALT and AST were observed (FIGS. 8A and 8B). Compared with semaglutide monotherapy, all combination groups had significantly greater improvements in ALT; the semaglutide+firsocostat and semaglutide+firsocostat+cilofexor groups also had significantly greater reductions in AST. Normalisation of ALT in patients with elevated levels at baseline was reported in 50% of patients in the semaglutide group and between 86% and 100% in the combination groups. Changes in other liver biochemistry parameters are summarized in Table 5 and Table 6.

TABLE 5
Change from baseline to week 24 in liver and metabolic biomarkers

| | | | | |
|---|---|---|---|---|
| | | | | Semaglutide 2.4 mg + |
| Semaglutide | Semaglutide 2.4 mg + | Semaglutide 2.4 mg + | Semaglutide 2.4 mg + | cilofexor 30 mg + |
| 2.4 mg | cilofexor 30 mg | cilofexor 100 mg | firsocostat 20 mg | firsocostat 20 mg |
| ( N = 21) | (N = 22) | (N = 22) | (N = 22) | (N = 21) |

Non-invasive markers of fibrosis

| Liver stiffness by MRE, kPa | n = 18 | | n = 19 | | n = 14 | | n = 18 | | n = 17 | |
|---|---|---|---|---|---|---|---|---|---|---|
| | −0.13 | (−0.40, 0.14) | −0.20 | (−0.46, 0.06) | 0.06 | (−0.24, 0.36) | −0.20 | (−0.47, 0.06) | −0.03 | (−0.30, 0.25) |
| ELF score | n = 16 | | n = 19 | | n = 17 | | n = 17 | | n = 17 | |
| | −0.56 | (−0.86, −0.27) | −0.46 | (−0.73, −0.19) | −0.47 | (−0.76, −0.19) | −0.59 | (−0.87, −0.30) | −0.42 | (−0.70, −0.13) |
| Patients with ≥ 0.5 reduction | 11 | (69) | 8 | (42) | 8 | (47) | 10 | (59) | 7 | (41) |
| in ELF, n (%) | | | | | | | | | | |
| FibroSure/FibroTest Score | n = 16 | | n = 19 | | n = 17 | | n = 18 | | n = 17 | |
| | 0 | (−0.04, 0.04) | −0.05 | (−0.09, −0.02) | −0.03 | (−0.07, 0.01) | −0.01 | (−0.05, 0.03) | −0.04 | (−0.09, 0) |

Biomarkers of Inflammation and hepatocellular apoptosis

| C-reactive protein, mg/dL | n = 16 | | n = 20 | | n = 17 | | n = 18 | | n = 17 | |
|---|---|---|---|---|---|---|---|---|---|---|
| | −0.23 | (−0.42, −0.04) | −0.13 | (−0.30, 0.04) | 0.05 | (−0.13, 0.24)* | −0.01 | (−0.19, 0.17) | −0.19 | (−0.38, −0.01) |
| CK-18 M30, U/L | n = 16 | | n = 20 | | n = 17 | | n = 18 | | n = 17 | |
| | −179 | (−252, −107) | −259 | (−323, −194) | −213 | (−284, −143) | −312 | (−381, −243)* | −247 | (−318, −176) |

Metabolic parameters

| Body weight, kg | n = 15 | | n = 20 | | n = 17 | | n = 18 | | n = 18 | |
|---|---|---|---|---|---|---|---|---|---|---|
| | −7.0 | (−9.9, −4.0) | −11.0 | (−13.5, −8.4)* | −9.9 | (−12.6, −7.1) | −8.3 | (−11.0, −5.6) | −7.7 | (−10.4, −4.9) |
| Body weight, %^{†} | −7.6 | (−9.2, −4.2) | −8.6 | (−13.3, −6.0) | −9.6 | (−14.7, −6.3) | −7.6 | (−11.7, −4.1) | −7.0 | (−10.3, −3.7) |
| Patients with ≥ 10 | 3 | (20%) | 9 | (45%) | 8 | (47%) | 5 | (28%) | 5 | (28%) |
| weight loss, n (%) | | | | | | | | | | |
| Body mass index, kg/m² | n = 15 | | n = 20 | | n = 17 | | n = 18 | | n = 18 | |
| | −2.6 | (−3.7, −1.6) | −4.0 | (−4.9, −3.2)* | −3.7 | (−4.7, −2.7) | −3.0 | (−4.0, −2.1) | −2.5 | (−3.5, −1.5) |
| Fasting plasma glucose, | n = 16 | | n = 20 | | n = 17 | | n = 18 | | n = 16 | |
| mg/dL | −31 | (−40, −23) | −22 | (−30, −14) | −18 | (−26, −10)* | −32 | (−40, −23) | −23 | (−31, −14) |
| HbA_{1c}, % | n = 16 | | n = 19 | | n = 17 | | n = 18 | | n = 17 | |
| | −1.0 | (−1.2, −0.7) | −1.1 | (−1.3, −0.9) | −1.0 | (−1.2, −0.8) | −1.2 | (−1.4, −1.0) | −1.0 | (−1.2, −0.8) |
| In patient with type 2 diabetes | n = 7 | | n = 10 | | n = 8 | | n = 12 | | n = 10 | |
| | −1.4 | (−1.8, −1.0) | −1.7 | (−2.0, −1.3) | −1.2 | (−1.6, −0.8) | −1.7 | (−2.0, −1.4) | −1.4 | (−1.8, −1.1) |

Data are least squares mean (95% CI) unless otherwise stated.
*p < 0.05 vs semaglutide alone.
^{†}Data for % weight loss are medians (Q1, Q3).
Least squares means, 95% CIs and p-values were estimated from the ANCOVA models of the change from baseline at week 24 adjusted for baseline value and baseline diabetes status.
CI, confidence interval;
CK, cytokeratin;
ELF, Enhanced Liver Fibrosis;
HbA_{1c}, haemoglobin A_{1c};
MRE, magnetic resonance elastography

TABLE 6
Changes from baseline to week 24 in additional liver biomarkers and metabolic parameters

| | | | | |
|---|---|---|---|---|
| | | | | Semaglutide 2.4 mg + |
| Semaglutide | Semaglutide 2.4 mg + | Semaglutide 2.4 mg + | Semaglutide 2.4 mg + | cilofexor 30 mg + |
| 2.4 mg | cilofexor 30 mg | cilofexor 100 mg | firsocostat 20 mg | firsocostat 20 mg |
| (n = 21) | (n = 22) | (n = 22) | (n = 22) | (n = 21) |

Markers of liver injury and function

| | | | | | | | | | | |
|---|---|---|---|---|---|---|---|---|---|---|
| ALP, U/L | −7 | (−20, 6) | −2 | (−13, 9) | 20 | (8, 32)* | 3 | (−8, 15) | 17 | (5, 29)* |
| GGT, U/L | −22 | (−38, −6) | −40 | (−54, −26) | −25 | (−41, −9) | −21 | (−36, −6) | −23 | (−38, −8) |
| Total bilirubin, mg/dL | 0 | (−0.1, 0.1) | 0 | (−0.1, 0.1) | 0 | (−0.1, 0.1) | 0 | (−0.1, 0.1) | −0.1 | (−0.2,0) |
| Direct bilirubin, mg/dL | 0.01 | (−0.02, 0.04) | −0.02 | (−0.04, 0.01) | −0.04 | (−0.07, −0.01) | −0.01 | (−0.04, 0.02) | −0.05 | (−0.08, −0.02)* |
| Albumin, g/dL | 0 | (−0.1, 0.1) | 0.1 | (0, 0.2) | 0 | (−0.1, 0.1) | 0 | (−0.1, 0.1) | −0.1 | (−0.2, 0) |
| International Normalized Ratio | 0 | (0, 0) | −0.1 | (−0.1, 0) | −0.1 | (−0.1,0)* | 0 | (−0.1, 0) | −0.1 | (−0.1, 0) |
| MELD score | 0 | (0, 1) | 0 | (0, 0) | 0 | (0, 0) | 0 | (−1, 0) | 0 | (−1, 0) |

Noninvasive markers of fibrosis and steatosis
ELF score components:

| | | | | | | | | | | |
|---|---|---|---|---|---|---|---|---|---|---|
| Hyaluronic acid, ng/mL | −16.3 | (−37.2, 4.6) | −16.4 | (−35.4, 2.5) | −11.2 | (−31.3, 9.0) | −12.6 | (−32.7, 7.6) | −12.9 | (−33.0, 7.2) |
| TIMP1, ng/mL | −28.8 | (−48.3, −9.4) | −41.4 | (−59.3, −23.5) | −15.0 | (−33.8, 3.9) | −43.4 | (−62.4, −24.4) | −29.1 | (−48.0, −10.2) |
| PIIINP, ng/mL | −2.8 | (−3.8, −1.7) | −3.5 | (−4.5, −2.6) | −2.4 | (−3.4, −1.4) | −3.2 | (−4.3, −2.2) | −2.6 | (−3.6, −1.6) |

FibroSure/FibroTest Score components:

| | | | | | | | | | | |
|---|---|---|---|---|---|---|---|---|---|---|
| α2 macroglobulin, mg/dL | 1 | (−12, 15) | 1 | (−11, 13) | 1 | (−12, 14) | 18 | (5, 31) | 1 | (−12, 14) |
| Haptoglobin, mg/dL | 4 | (−19, 28) | 13 | (−8, 34) | 19 | (−5, 42) | 25 | (2, 48) | 34 | (11, 57) |
| Apolipoprotein A1, mg/dL | −9 | (−18, −1) | −17 | (−24, −10) | −19 | (−27, −11) | −13 | (−21, −5) | −19 | (−27, −11) |

Markers of glycemic control

| | | | | | | | | | | |
|---|---|---|---|---|---|---|---|---|---|---|
| HOMA-IR | −3.5 | (−5.3, −1.7) | −3.9 | (−5.5, −2.2) | −2.3 | (−4.1, −0.6) | −3.8 | (−5.5, −2.1) | −2.5 | (−4.3, −0.7) |
| Fasting insulin, uIU/mL | −8.5 | (−14.6, −2.5) | −10.6 | (−16.0, −5.2) | −6.3 | (−12.1, −0.5) | −8.2 | (−13.9, −2.5) | −4.6 | (−10.6, 1.5) |

Markers of renal function

| | | | | | | | | | | |
|---|---|---|---|---|---|---|---|---|---|---|
| eGFR, mL/min/1.73 m² | −6.6 | (−13.4, 0.3) | −2.1 | (−8.1, 3.8) | −2.6 | (−9.0, 3.8) | −2.0 | (−8.3, 4.3) | 1.1 | (−5.3, 7.6) |
| Creatinine, mg/dL | 0.07 | (0, 0.14) | 0.02 | (−0.04, 0.08) | 0.05 | (−0.01, 0.11) | 0 | (−0.06, 0.06) | −0.02 | (−0.08, 0.04) |

Data are least squares mean (95% CI) based on ANCOVA models adjusted for baseline and type 2 diabetes status unless otherwise stated.
*p < 0.05 vs semaglutide alone.
ALP, alkaline phosphatase;
APRI, Aspartate Aminotransferase to Platelet Ratio Index;
CI, confidence interval;
eGFR, estimated glomerular filtration rate;
GGT, gamma-glutamyl transferase;
HOMA-IR, homeostasis model assessment of insulin resistance;
MELD, Model of End-Stage Liver Disease;
NAFLD, non-alcoholic fatty liver disease;
PIIINP, procollagen III amino terminal propeptide;
TIMP1, tissue inhibitor of metalloproteinase

### NITs of Fibrosis, Other Biomarkers, and Quality-of-Life

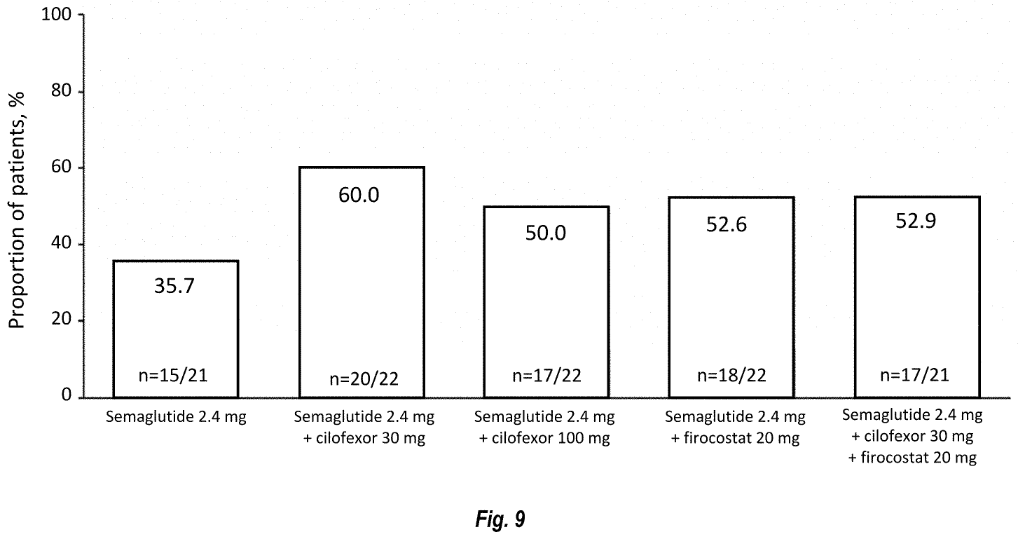

Reductions in liver stiffness by TE were similar across treatment groups; LS mean changes ranged from −2.29 to −3.74 kPa (FIG. 8C). The largest reductions from baseline to week 24 were observed in the two firsocostat-containing groups (semaglutide+firsocostat, −3.50 kPa; semaglutide+firsocostat+cilofexor, −3.74 kPa). The proportions of patients with a ≥25% relative reduction in liver stiffness by TE from baseline to week 24 were higher in the combination groups (50% to 60%) compared with the semaglutide group (36%) (FIG. 9). Liver stiffness by MRE did not change between baseline and week 24 and no significant differences were observed between treatment groups (Table 5).

At week 24, statistically significant reductions from baseline in ELF score were observed in all groups (from 0.42 to 0.59 units); no significant differences between groups were observed (Table 5). Changes in individual ELF components are reported in Table 6. In a post-hoc analysis, all combinations except semaglutide+cilofexor 100 led to significantly greater improvements in FAST score compared to semaglutide (FIG. 8D). Reductions in FibroSure/FibroTest from baseline to week 24 was also observed; however, there were no significant differences between the semaglutide group and the combination therapies (Table 5; Table 6).

Reductions from baseline in serum levels of CK-18 M30, a biomarker of hepatocyte apoptosis, were observed in all treatment groups at week 24; these reductions were significantly greater in the semaglutide+firsocostat group versus semaglutide monotherapy (p=0.0102). With the exception of the semaglutide+cilofexor 100-treated patients, CRP declined in all treatment groups.

Relative reductions from baseline to week 24 in body weight were similar across groups (−7.6 with semaglutide, −7.0 to −9.6% with combinations) (Table 5). Proportions of patients with ≥10% weight loss at week 24 ranged from 20% with semaglutide to 28% to 47% with combinations. Decreases in HbA_{1c} at week 24 were similar across the treatment groups (−1.0 to −1.2%) (Table 5). No consistent pattern of improvement in health-related quality-of-life across treatment groups was observed for any of the patient-reported outcomes (Table 7).

TABLE 7
Changes from baseline to week 24 in quality-of-life scores

| | | | | | Semaglutide 2.4 mg + |
|---|---|---|---|---|---|
| | Semaglutide | Semaglutide 2.4 mg + | Semaglutide 2.4 mg + | Semaglutide 2.4 mg + | cilofexor 30 mg + |
| | 2.4 mg | cilofexor 30 mg | cilofexor 100 mg | firsocostat 20 mg | firsocostat 20 mg |
| | (n = 16) | (n = 21) | (n = 18) | (n = 19) | (n = 19) |
| CLDQ-NAFLD | 0.2 (−0.6, 0.6) | −0.1 (−0.4, 0.3) | 0.1 (−0.3, 0.5) | 0.0 (−0.8, 1.0) | 0.3 (−0.5, 0.5) |
| EQ-5D, n (%) | | | | | |
| Mobility: | | | | | |
| Improvement | 0 | 4 (19.0) | 1 (5.6) | 4 (21.1) | 2 (10.5) |
| No change | 16 (100) | 15 (71.4) | 11 (61.1) | 13 (68.4) | 14 (73.7) |
| Worsening | 0 | 2 (9.5) | 6 (33.3) | 2 (10.5) | 3 (15.8) |
| Self-care | | | | | |
| Improvement | 1 (6.3) | 1 (4.8) | 0 | 1 (5.3) | 1 (5.3) |
| No change | 14 (87.5) | 19 (90.5) | 18 (100) | 16 (84.2) | 18 (94.7) |
| Worsening | 1 (6.3) | 1 (4.8) | 0 | 2 (10.5) | 0 |
| Usual activities | | | | | |
| Improvement | 1 (6.3) | 4 (19.0) | 1 (5.6) | 1 (5.3) | 2 (10.5) |
| No change | 12 (75.0) | 15 (71.4) | 11 (61.1) | 15 (78.9) | 14 (73.7) |
| Worsening | 3 (18.8) | 2 (9.5) | 6 (33.3) | 3 (15.8) | 3 (15.8) |
| Pain/discomfort | | | | | |
| Improvement | 3 (18.8) | 4 (19.0) | 5 (27.8) | 5 (26.3) | 4 (21.1) |
| No change | 10 (62.5) | 12 (57.1) | 11 (61.1) | 11 (57.9) | 10 (52.6) |
| Worsening | 3 (18.8) | 5 (23.8) | 2 (11.1) | 3 (15.8) | 5 (26.3) |
| Anxiety/depression | | | | | |
| Improvement | 5 (31.3) | 2 (9.5) | 3 (16.7) | 5 (26.3) | 6 (31.6) |
| No change | 8 (50.0) | 13 (61.9) | 15 (83.3) | 9 (47.4) | 10 (52.6) |
| Worsening | 3 (18.8) | 6 (28.6) | 0 | 5 (26.3) | 3 (15.8) |
| EQ VAS | 5 (0, 8) | 8 (0, 15) | 0 (−5, 7) | 0 (−10, 10) | 5 (0, 10) |

Data are median (Q1, Q3) change for CLDQ-NAFLD and EQ VAS.
CLDQ-NAFLD, chronic liver disease questionnaire - nonalcoholic fatty liver disease;
EQ-5D, EuroQol 5 dimensions,
EQ VAS = EuroQol visual analogue scale

### Discussion

In this randomized trial of patients with NASH and mild-to-moderate fibrosis, treatment with semaglutide in combination with cilofexor and/or firsocostat was well tolerated and associated with improvements in hepatic steatosis as measured by MRI-PDFF and CAP, liver stiffness as measured by TE, FAST score, and serum ALT and AST, when compared with semaglutide alone.

The tolerability of combinations including semaglutide, cilofexor, and/or firsocostat was similar to that of semaglutide monotherapy. Most adverse events were mild-to-moderate in severity, treatment discontinuation due to adverse events was infrequent, and only two patients had serious adverse events. The most frequent adverse events were gastrointestinal, in particular, nausea, diarrhea, constipation, and decreased appetite. Discontinuations due to adverse events and rates of gastrointestinal events were not increased with combinations compared with semaglutide alone. The incidence of pruritus, an adverse event associated with both NASH and FXR agonist therapy [Patel et al. Hepatology. 2020; 72(1):58-71; Younossi et al. Hepatol Commun. 2020; 4(11):1637-1650], was low, occurring in just five of 65 cilofexor-treated patients (7.7%). The pruritus events were mild and none led to discontinuation of treatment.

Treatment with both FXR agonists and ACC inhibitors has been associated with increases in serum lipids, including LDL cholesterol and triglycerides, respectively [Loomba et al. Gastroenterology. 2018; 155(5):1463-1473; Younossi et al. Lancet. 2019; 394(10215):2184-2196; Patel et al. Hepatology. 2020; 72(1):58-71; Loomba et al. Hepatology. 2020 Nov. 10. doi: 10.1002/hep.31622]. Previous studies have shown that ACC inhibitor-related hypertriglyceridemia is greatest in patients with pre-existing dyslipidemia and can be mitigated via the use of fibrates and/or fish oil [Loomba et al. Gastroenterology. 2018; 155(5):1463-1473; Loomba et al. Hepatology. 2020 Nov. 10. doi: 10.1002/hep.31622]. In contrast, improvements in serum lipids have been observed with semaglutide treatment [Newsome. Lancet Gastroenterol Hepatol. 2020 April; 5(4):362-373]. Total cholesterol, LDL cholesterol, and triglycerides decreased with semaglutide monotherapy. These benefits were generally reduced with the addition of cilofexor and/or firsocostat to semaglutide. Firsocostat-related hypertriglyceridemia was relatively mitigated by the addition of semaglutide compared with data from prior studies [Loomba et al. Gastroenterology. 2018; 155(5):1463-1473; Loomba et al. Hepatology. 2020 Nov. 10. doi: 10.1002/hep.31622].

Compared with semaglutide monotherapy, combinations including cilofexor and/or firsocostat resulted in greater reductions in hepatic steatosis assessed by MRI-PDFF and CAP. These beneficial effects were observed as early as 12 weeks after treatment initiation. Given that the 2.4 mg target dose of semaglutide was not reached until week 17 onwards, further reductions in hepatic steatosis may be expected with longer-term treatment, as was observed with semaglutide monotherapy between 24 and 48 weeks in a previous placebo-controlled trial in 67 patients with NAFLD [Flint et al 2020]. In addition, more patients treated with combinations achieved reductions of ≥30% and ≥50% in MRI-PDFF compared with semaglutide. Eighty-six percent of patients treated with combinations had a ≥30% improvement in MRI-PDFF and 40% had normalization of liver fat (<5%) after 24 weeks of treatment. These improvements are among the highest reported in a NASH trial and are of a magnitude that has been associated with an increased likelihood of histologic response, including NASH resolution and fibrosis improvement [Loomba et al. Hepatology. 2020c; 72(4):1219-1229; Loomba et al. J Hepatol 20201; 73:S56 (AS077); Stine et al. Clin Gastroenterol Hepatol. 2020:S1542-3565(20)31220-9].

All combinations were associated with improvements in liver biochemistry that were similar or greater than those observed with semaglutide monotherapy. Greater reductions in ALT were observed in all combination groups, and normalization of ALT occurred in 86% to 100% of patients across these groups. Approximately 60% to 70% of patients had a ≥17 U/L reduction in ALT at week 24 (data not shown), a threshold that has been associated with histological improvement [Loomba et al. Hepatology. 2020c; 72(4):1219-1229]. Moreover, reductions from baseline in CK-18 M30, a biomarker of hepatocellular apoptosis, as well as CRP, a biomarker of inflammation, were observed.

While necroinflammatory activity is the key driver of fibrosis progression in NASH, fibrosis is the primary determinant of liver-related morbidity and mortality [Angulo et al. Gastroenterology. 2015; 149(2):389-97.e10; Sanyal et al. Hepatology. 2019; 70(6):1913-1927]. Beneficial effects on multiple NITs of fibrosis were observed with semaglutide alone and in combination with cilofexor and firsocostat. For example, ELF improvements were similar across all groups and ranged from 0.42 to 0.59 units. Similarly, improvements in liver stiffness by TE were observed in all groups, with a trend towards greater reductions in firsocostat-treated patients. Moreover, a higher proportion of patients in combination groups had a ≥25% relative reduction in liver stiffness. While the trial lacked liver biopsy for histologic confirmation of anti-fibrotic effects, changes in ELF and liver stiffness of these magnitudes have been associated with reduced rates of disease progression in patients with advanced fibrosis due to NASH [Sanayl et al 2019; Harrison et al 2020]. In addition, we observed significantly greater reductions in FAST score, a combination of liver stiffness and CAP by TE and serum AST [Newsome et al. Hepatol. 2020 April; 5(4):362-373; Boursier et al. J Hepatol 2020; 73: AS075], in the combination groups versus semaglutide alone. Based on the utility of FAST for identifying NASH patients with fibrosis (≥F2) and active necroinflammatory activity (NAS≥4), these findings add support to the potential of these combination therapies.

Treatment with semaglutide, alone and in combination with cilofexor and/or firsocostat, also led to improvements in metabolic parameters, including body weight and glycaemic control. Body weight declined in all groups, with percentage weight loss of 7% to 9.6% across groups. This magnitude of weight loss has been associated with histological improvement in NASH [Vilar-Gomez Gastroenterology. 2015; 149(2):367-78.e5]. Similar weight loss across the groups indicates that the greater reductions in liver fat, FAST, ALT, and AST with combinations are not mediated by additional body weight reduction with cilofexor and/or firsocostat, and support the complementarity of FXR agonism and ACC inhibition to GLP-1 receptor agonism with semaglutide. The changes in body weight observed in this trial are consistent with previous observations regarding semaglutide in type 2 diabetes and obesity [Sorli et al. Lancet Diabetes Endocrinol. 2017; 5(4):251-260; O'Neil et al. Lancet. 2018; 392(10148):637-649]. In the phase 2 trial of semaglutide in NASH, a mean weight loss of 13% was observed after 72 weeks of therapy with the highest dose of semaglutide (0.4 mg once daily) [Newsome et al. Lancet Gastroenterol Hepatol. 2020 April; 5(4):362-373], which is similar to the 2.4 mg weekly target dose utilized in the current trial. Based on these data, additional weight loss would be expected with longer-term semaglutide therapy, especially given the target dose of semaglutide was not reached until week 17 of the trial. All groups experienced improvements in glycaemic parameters, including fasting plasma glucose and HbA_{1c}, the latter ranging from −1.2 to −1.7% among patients with type 2 diabetes. In summary, in this phase 2 trial, semaglutide in combination with cilofexor and/or firsocostat was well tolerated in patients with mild-to-moderate fibrosis due to NASH. Combination treatments resulted in greater improvements in hepatic steatosis, liver biochemistry, and several metabolic and hepatic biomarkers, including NITs of fibrosis, than achieved with semaglutide alone.

### Example 2: Clinical Study Evaluating Fixed-Dose Combination of Cilofexor and Firsocostat in Subjects with NASH

A. Overview of Study Design

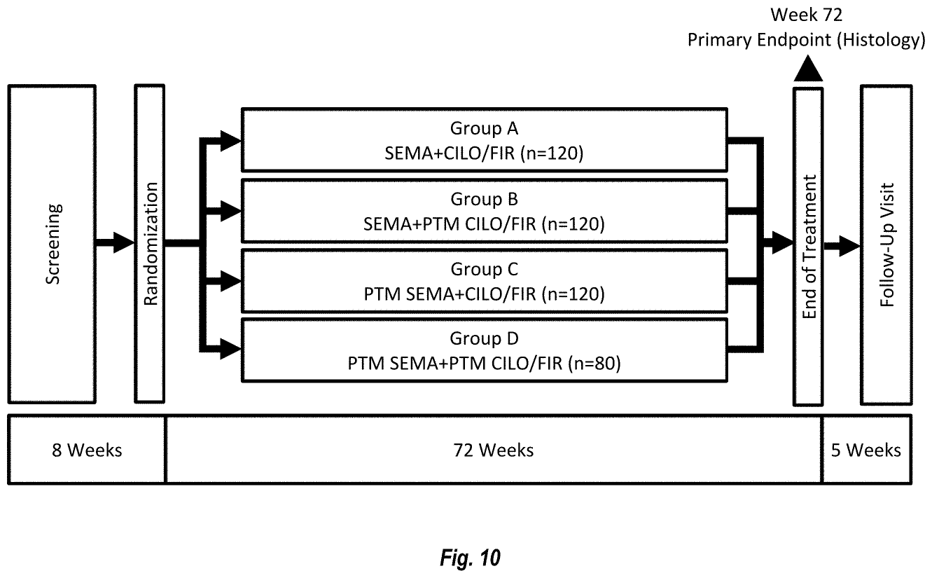

This is a Phase 2, randomized, double-blind, double-dummy, placebo-controlled study evaluating the efficacy and safety of semaglutide (SEMA), cilofexor/firsocostat (CILO/FIR), and their combination in subjects with compensated cirrhosis due to NASH. Subjects meeting the study's entry criteria are randomly assigned in a 3:3:3:2 ratio to 1 of 3 active treatment groups (SEMA+CILO/FIR, SEMA alone, CILO/FIR alone) or placebos-to-match (PTM), as shown in FIG. 10. Randomization are stratified by the presence or absence of type 2 diabetes as determined by medical history or based on screening laboratory values if previously undiagnosed (ie, HbA1c≥6.5% or fasting plasma glucose ≥126 mg/dL, confirmed on repeat testing), and by Enhanced Liver Fibrosis (ELF) score (≥11.30 or <11.30 during screening). Subjects are treated for 72 weeks. Total study duration is up to 85 weeks, including up to 8 weeks for screening, a 72-week treatment period, and a 5-week follow-up period.

B. Subject Population

This study will enroll approximately 440 subjects with compensated cirrhosis due to NASH. Subjects who discontinue before the end of study are not be replaced. Subjects must meet all of the following inclusion criteria to be eligible for participation in this Study.

### Inclusion Criteria

1. Men and women between 18-80 years of age, inclusive, based on the date of the screening visit
2. Cirrhosis (F4) due to NASH as defined by one of the following:
   a) a historical liver biopsy within 180 days of screening that, in the opinion of the central pathologist, is evaluable and consistent with cirrhosis (F4) and NASH (defined as the presence of steatosis Grade ≥1, hepatocellular ballooning Grade ≥1, and lobular inflammation Grade ≥1, according to NAS)
OR
   b) In subjects without a qualifying historical liver biopsy, if FibroScan ≥9.9 kPa at screening, a screening liver biopsy may be performed. The screening liver biopsy must, in the opinion of the central pathologist, be evaluable and meet histologic criteria as specified in inclusion criterion 3a).
OR
   c) In subjects with a historical liver biopsy completed more than 180 days prior to screening that is consistent with cirrhosis (F4) and NASH, as determined by a local reader, a screening liver biopsy may be performed. The screening liver biopsy must, in the opinion of the central pathologist, be evaluable and meet histologic criteria as specified in inclusion criterion 3a).
3. The following laboratory parameters at screening, as determined by the central laboratory:
   a) Estimated glomerular filtration rate (eGFR)≥30 mL/min/1.73 m2, as calculated by the Modification of Diet in Renal Disease (MDRD) equation to estimate creatinine clearance (CLcr)
   b) HbA1c≤10% (or serum fructosamine≤400 umol/L if HbA1c is not quantifiable)
   c) Hemoglobin >10.6 g/dL
   d) INR≤1.4, unless due to therapeutic anticoagulation
   e) Total bilirubin ≤1.3×ULN (unless due to an alternative etiology such as Gilbert's syndrome or hemolytic anemia)
   f) Platelet count≥125,000/uL
   g) Serum triglyceride level≤250 mg/dL. If initial screening value is >250 mg/dL, triglycerides may be retested during the screening period. Fasting serum triglycerides must be confirmed to be ≤250 mg/dL prior to Day 1. Management of hypertriglyceridemia may be initiated or modified at investigator discretion during the screening period.
   h) ALT<5 XULN
4. BMI≥23 kg/m2 at screening

### Exclusion Criteria

Subjects who meet any of the following exclusion criteria are not eligible to be enrolled in this study:
1. Any history of decompensated liver disease in the opinion of the investigator, including clinically relevant ascites, hepatic encephalopathy (HE), or variceal bleeding
2. Child-Pugh (CP) score >6 at screening, unless due to an alternative etiology such as Gilbert's syndrome or therapeutic anticoagulation
3. Model for End-stage Liver Disease (MELD) score >12 at screening, unless due to an alternative etiology such as therapeutic anticoagulation
4. Chronic HBV infection (HBsAg positive)
5. Chronic HCV infection (HCV antibody and HCV RNA positive). Subjects cured of HCV infection less than 2 years prior to the screening visit are not eligible.
6. Other causes of liver disease based on medical history and/or central pathologist review of liver histology, including but not limited to: alcoholic liver disease, autoimmune disorders (e.g., PBC, PSC, autoimmune hepatitis), drug-induced hepatotoxicity, Wilson disease, clinically significant iron overload, or alpha-1-antitrypsin deficiency
7. History of liver transplantation
8. Current or prior history of HCC
9. HIV infection
10. Weight loss >10% within 180 days of screening, or >5% between the date of the biopsy used for eligibility and the date of screening
11. Any weight reduction surgery or procedure in the 2 years prior to screening or malabsorptive weight loss surgery (e.g., Roux-en-Y or distal gastric bypass) at any time prior to screening
12. History of intestinal resection that could result in malabsorption of study drug
13. Planned coronary, carotid, or peripheral artery intervention or unstable cardiovascular disease in the opinion of the investigator, including any of the following:
   a) Unstable angina, myocardial infarction, coronary artery bypass graft surgery, or coronary angioplasty within 180 days prior to screening
   b) Transient ischemic attack or cerebrovascular accident within 180 days prior to screening
   c) Symptomatic valvular heart disease or hypertrophic cardiomyopathy
   d) Symptomatic congestive heart failure
   e) Uncontrolled or recurrent ventricular tachycardia or arrhythmia requiring an automatic implantable cardioverter defibrillator. Stable, controlled atrial fibrillation is allowed.
   f) An emergency room visit or hospitalization for confirmed cardiovascular disease within 180 days prior to screening
14. History of uncontrolled chronic pulmonary disease in the opinion of the investigator (e.g., chronic obstructive pulmonary disease, interstitial lung disease) within 180 days prior to screening
15. Men who habitually drink greater than 21 units/week of alcohol or women who habitually drink greater than 14 units/week of alcohol (one unit is equivalent to 12 oz/360 mL of beer, a 4 oz/120 mL glass of wine, or 1 oz/30 mL of hard liquor)
16. Positive urine drug screen for amphetamines, cocaine, or opiates (e.g., heroin, morphine) at screening, unless due to a prescription medication (e.g., oxycodone, methylphenidate) and the prescription and diagnosis are reviewed and approved by the investigator. Subjects on stable methadone or buprenorphine maintenance treatment for at least 180 days prior to screening may be included in the study
17. Use of any prohibited concomitant medication prior to enrollment
   a) Subjects on vitamin E regimen ≥800 IU/day, or pioglitazone, must be on a stable dose in the opinion of the investigator for at least 180 days prior to the historical or screening liver biopsy
   b) Subjects taking antidiabetic medications must be on a stable dose, in the opinion of the investigator, for at least 90 days prior to the historical or screening liver biopsy
18. History of malignancy within 5 years of screening with the following exceptions:
   a) Adequately treated carcinoma in situ of the cervix
   b) Adequately treated basal or squamous cell cancer or other localized nonmelanoma skin cancer
19. For subjects with type 2 diabetes diagnosed prior to the date of the screening visit OR based on screening visit results (HbA1c≥6.5% or fasting plasma glucose ≥126 mg/dL, confirmed on repeat testing), subjects must have no evidence of uncontrolled and potentially unstable retinopathy or maculopathy as determined by a fundoscopic examination performed starting 90 days prior to screening visit date through Day 1. If there has been worsening of the subject's visual function since a historical fundoscopic examination in the opinion of the investigator, then the fundoscopic examination must be repeated prior to Day 1 for eligibility. Pharmacological pupil dilation is a requirement unless using a digital fundus photography camera specified for nondilated examination.
20. Acute pancreatitis within 180 days prior to the date of the screening visit
21. History or presence of chronic pancreatitis
22. Presence or history of type 1 diabetes
23. Personal or first-degree relative(s) history of multiple endocrine neoplasia type 2 or medullar thyroid carcinoma
24. Treatment with GLP-1 RAs (including semaglutide) in the period from 90 days prior to the screening visit and from 90 days prior to the date of the historical qualifying liver biopsy (if applicable)

C. Drug Products

### Semaglutide

Semaglutide solution for injection has the composition shown above in Table 1. Semaglutide solution for injection is a colorless or almost colorless liquid, free from turbidity and essentially free from particulate matter. The PDS290 pen-injector (FlexTouch®) for semaglutide is a dial-a-dose prefilled device integrated with a 3 mL cartridge filled with semaglutide 3.0 mg/mL. The pen-injector can deliver doses from 1 to 80 dose steps in increments of 1. The user can dial up and down in order to adjust a dose.

### Cilofexor and Fisocostat

Cilofexor/firsocostat 30 mg/20 mg tablets are provided as a fixed-dose combination tablets as described herein.

D. Dosage and Administration

Subjects take semaglutide subcutaneously with a PDS290 pen-injector at approximately the same time each week. Subjects take a cilofexor/firsocostat 30 mg/20 mg tablet (if applicable) at approximately the same time each day, with or without food, swallowed whole with water. Subjects taking a concomitant acid reducing agent, including H2-receptor antagonists, should be instructed to take the cilofexor/firsocostat 30 mg/20 mg tablet with food.

Study drug dosing and administration is as follows, based on treatment group randomization as summarized in FIG. 10:
Group A: Semaglutide 3.0 mg/mL administered subcutaneously with prefilled pen injector once weekly and one cilofexor/firsocostat 30 mg/20 mg tablet administered orally once daily, both without regard to food
Group B: Semaglutide 3.0 mg/mL administered subcutaneously with prefilled pen injector once weekly and one PTM cilofexor/firsocostat 30 mg/20 mg tablet, administered orally once daily, both without regard to food
Group C: PTM semaglutide 3.0 mg/mL administered subcutaneously with prefilled pen injector once weekly and one cilofexor/firsocostat 30 mg/20 mg tablet administered orally once daily, both without regard to food
Group D: PTM semaglutide 3.0 mg/mL administered subcutaneously with prefilled pen injector once weekly and one PTM cilofexor/firsocostat 30 mg/20 mg tablet administered orally once daily, both without regard to food

After randomization, semaglutide is initiated with a starting value of 8 (0.24 mg) as shown on the dose counter of the prefilled pen injector for the first 4 weeks (4 doses), and subsequently the value is increased every 4 weeks. The semaglutide dose escalation scale is shown in Table 8.

TABLE 8
Semaglutide Dose Escalation Schedule

| | Dose | Volume | |
|---|---|---|---|
| Product | (mg) | (μL) | Duration |
| Semaglutide 3.0 mg/mL | 0.24 | 80 | Day 1* up to Week 4 |
| Semaglutide 3.0 mg/mL | 0.50 | 170 | Week 4 up to Week 8 |
| Semaglutide 3.0 mg/mL | 1.0 | 340 | Week 8 up to Week 12 |
| Semaglutide 3.0 mg/mL | 1.7 | 570 | Week 12 up to Week 16 |
| Semaglutide 3.0 mg/mL | 2.4 | 800 | Week 16 up to Week 72 |

*Subject takes first dose of study drug on-site at Day 1.

If a subject does not tolerate the planned 4-week dose-escalation regimen due to GI AEs or for other reasons as judged by the investigator, the subject may stay longer at any dose level.

E. Assessments

### Clinical Laboratory Analytes

In general, subjects fast (no food or drink, except water) for approximately 10 hours prior to the blood sample collection.

The following chemistry analytes are evaluated: alanine aminotransferase (ALT), aspartate aminotransferase (AST), albumin, alkaline phosphatase (ALP), bicarbonate, blood urea nitrogen (BUN), calcium, chloride, creatinine (reflex to serum enzymatic creatinine, as applicable), lactate dehydrogenase, magnesium, phosphorus, potassium, sodium, total and direct bilirubin, total protein, uric acid, and gamma-glutamyl transferase (GGT).

The following hematology factors are measured: Hematocrit (Hct), hemoglobin (Hb), platelet count, red blood cell count (RBC), white blood cell count (WBC) with differential (absolute and percentage) including lymphocytes, monocytes, neutrophils, eosinophils, basophils, and mean corpuscular volume (MCV). The coagulation panel includes INR, prothrombin time (PT), and partial thromboplastin time (PTT).

The following glycemic panel is evaluated: insulin, homeostasis model assessment of Insulin resistance (HOMA-IR based on fasting glucose and insulin), and C-peptide.

The following Lipid Panel is evaluated: triglycerides, total cholesterol, high density lipids (HDL), non-HDL, low density lipids (LDL) and very low density lipids (VLDL) by Friedewald calculation.

Additional tests include HbA1c (reflex to serum fructosamine, as applicable), HIV-1 (reflex to HIV-1 RNA), HBV (HBsAg), HCV (reflex to HCV RNA) serology, homeostasis model assessment of Insulin resistance (HOMA-IR, based on fasting glucose and insulin), eGFR as calculated by MDRD, urine drug screen (for amphetamines, cocaine, opiates), serum pregnancy test, serum follicle-stimulating hormone (FSH) test, reflex direct LDL (if triglycerides are >400 mg/dL), CK, and optional genomic testing; biomarker tests including but not limited to C-reactive protein, NMR LipoProfile®, ELF, CK18 M30, CK18 M65, ProC3, CTXIII, total serum bile acids, apolipoproteins, and, potentially, levels of hepatic genes and proteins; urine samples for microalbumin, creatinine, microalbumin/creatinine ratio; at screening for amphetamines, cocaine, methadone, and opiates; urine pregnancy test (reflex to serum beta human chorionic gonadotropin), and stored for future biomarker testing

For a pharmacokinetic assessments single PK plasma samples are collected and archived for PK analysis of cilofexor and firsocostat (and their metabolites, as applicable). Samples are collected at Week 4 (15 minutes to 3 hours postdose), Week 24 (anytime), Week 48 (predose), Week 60 (15 minutes to 3 hours postdose) and Week 72 (predose). For PK sampling at Weeks 4, 48, 60, and 72, subjects should be reminded not to take their oral study drug until advised to do so at their clinic visit.

For subjects with type 2 diabetes (from medical history or from Screening Hemoglobin A1c≥6.5%), a fundus exam is performed at Screening. Fundus examinations require pharmacological dilation of both pupils or the use of a digital fundus photography camera specified for non-dilated examination.

### Clinical Liver Assessments

MELD and CP scores are derived from the central laboratory values obtained at each visit. MELD is calculated using the following formula:

MELD score=10*([0.378*ln(total bilirubin mg/dL)]+[1.12*ln(INR)]+[0.957*ln(serum creatinine mg/dL)]+0.643)

The Child-Pugh (CP) score is used to assess the prognosis of chronic liver disease, primarily cirrhosis.

### Creatinine Clearance/eGFR

Estimated glomerular filtration rate (eGFR) is determined by creatinine clearance, which is calculated by the Modification of Diet in Renal Disease (MDRD) Study equation:

eGFR(mL/min/1.73 m²)=175×Serum Creatinine^{-1.154}×(Age)^{-0.203}×(1.212 if African American)×(0.742 if female).

Serum creatinine in μmol/L is rounded to zero decimal places and converted to mg/dL by multiplying by 0.01131 prior to applying the formula. Creatinine in mg/dL is rounded to 2 decimal places prior to applying formula.

### FibroScan®

Liver Stiffness is measured by transient elastography (FibroScan®). FibroScan examinations are performed at the Screening Visit Weeks 24, 48, and 72 and median liver stiffness in kilopascals (kPa), interquartile range/median value (IQR/M), and success rate (number of valid shots/total number of shots) are assessed. Where available, the median CAP and the interquartile range of CAP values are recorded from FibroScan® examinations.

### Liver Biopsy

A liver biopsy specimen of at least 2.0 cm in length should be acquired when possible to ensure accurate staging of fibrosis and other histological parameters. If a screening or Week 72 liver biopsy is deemed unevaluable by the central pathologist, it may be repeated. Week 72 liver biopsy results are blinded to the investigator and subject.

### Ultrasound

Abdominal ultrasound for hepatocellular carcinoma (HCC) surveillance is performed at the screening visit, though historical ultrasound within 90 days of the screening visit is acceptable. Abdominal ultrasounds should be performed again at Weeks 24, 48, and 72, and may be performed at the ET visit at the discretion of the investigator.

### ECG

Standard 12-lead electrocardiogram (ECG) assessments are performed at Screening and the Day 168 (Week 24) visit. The Investigator reviews the ECGs for any clinically significant abnormalities to ensure subject safety.

In addition patient-reported outcome measures may be assessed using standard questionnaires. Patients may also receive lifestyle counseling and counseling regarding adherence to the study procedures.

F. Endpoints

The primary objective of this study is to evaluate whether the combination of semaglutide with cilofexor/firsocostat causes fibrosis improvement and NASH resolution in subjects with compensated cirrhosis due to NASH.

The secondary objectives of this study are as follows:
to confirm the contribution of semaglutide to NASH resolution in subjects treated with the combination of semaglutide and cilofexor/firsocostat by comparing with subjects treated with cilofexor/firsocostat alone;
to confirm the contribution of cilofexor/firsocostat to fibrosis improvement in subjects treated with the combination of semaglutide and cilofexor/firsocostat by comparing with subjects treated with semaglutide alone.

The exploratory objectives of this study are as follows:
to evaluate the safety and tolerability of semaglutide, cilofexor/firsocostat, and their combination in subjects with compensated cirrhosis due to NASH;
to evaluate whether the combination of semaglutide and cilofexor/firsocostat causes both fibrosis improvement and NASH resolution;
to evaluate whether semaglutide or cilofexor/firsocostat alone causes fibrosis improvement and/or NASH resolution;
to evaluate whether semaglutide, cilofexor/firsocostat, or their combination causes improvement in hepatic steatosis, hepatocellular ballooning, and/or lobular inflammation as measured by the nonalcoholic fatty liver disease (NAFLD) Activity Score (NAS);
to evaluate whether semaglutide, cilofexor/firsocostat, or their combination causes improvement in histological parameters of NASH including fibrosis and necroinflammatory activity, as measured by a machine learning approach;
to evaluate whether semaglutide, cilofexor/firsocostat, or their combination leads to improvement in noninvasive markers of fibrosis including liver stiffness measured by FibroScan and the ELF test score;
to evaluate whether semaglutide, cilofexor/firsocostat, or their combination causes improvement in liver steatosis measured by CAP by FibroScan;
to evaluate whether semaglutide, cilofexor/firsocostat, or their combination leads to improvement in markers of liver injury and function including ALT, AST, bilirubin, and GGT;
to evaluate the effect of semaglutide, cilofexor/firsocostat, and their combination on metabolic parameters and risk factors for cardiovascular disease, including insulin resistance, hyperlipidemia, obesity, and blood pressure;
to evaluate the effect of semaglutide, cilofexor/firsocostat, and their combination on patient-reported outcome measures (PROs);
to evaluate the pharmacokinetics of semaglutide, cilofexor/firsocostat, and their combination in subjects with compensated cirrhosis due to NASH.

The coprimary endpoints are:
≥1-stage improvement in fibrosis (according to the NASH CRN classification) without worsening of NASH (defined as a ≥1-point increase in hepatocellular ballooning or lobular inflammation) at Week 72 in the semaglutide+cilofexor/firsocostat versus placebo groups;
NASH resolution (defined as lobular inflammation of 0 or 1 and hepatocellular ballooning of 0) at Week 72 in the semaglutide+cilofexor/firsocostat versus placebo groups.

The secondary endpoints of this study are as follows:
NASH resolution at Week 72 in the semaglutide+cilofexor/firsocostat versus subjects treated with cilofexor/firsocostat alone;
≥1-stage improvement in fibrosis (according to the NASH CRN classification) without worsening of NASH at Week 72 in semaglutide+cilofexor/firsocostat versus subjects treated with semaglutide alone.

The exploratory endpoints of interest are as follows:
NASH resolution at Week 72 in subjects treated with semaglutide or cilofexor/firsocostat versus placebo;
≥1-stage improvement in fibrosis without worsening of NASH at Week 72 in subjects treated with semaglutide or cilofexor/firsocostat, versus placebo;
NASH resolution and ≥1-stage improvement in fibrosis at Week 72 in subjects treated with semaglutide, cilofexor/firsocostat, or their combination, versus placebo;
change from baseline at Week 72 in NAS and individual components of steatosis, lobular inflammation, hepatocellular ballooning, and other histologic features in subjects treated with semaglutide, cilofexor/firsocostat, or their combination versus placebo;
change from baseline at Week 72 in histological parameters of NASH including fibrosis and necroinflammatory activity, as measured by a machine learning approach in subjects treated with semaglutide, cilofexor/firsocostat, or their combination versus placebo;
change from baseline at Week 72 in liver stiffness as measured by FibroScan in subjects treated with semaglutide, cilofexor/firsocostat, or their combination versus placebo;
change from baseline at Week 72 in ELF test score in subjects treated with semaglutide, cilofexor/firsocostat, or their combination versus placebo;
change from baseline at Week 72 in liver steatosis measured by CAP by FibroScan in subjects treated with semaglutide, cilofexor/firsocostat, or their combination versus placebo;
change from baseline at Week 72 in markers of liver injury and function including ALT, AST, bilirubin, and GGT in subjects treated with semaglutide, cilofexor/firsocostat, or their combination versus placebo;
change from baseline at Week 72 in metabolic parameters and risk factors for cardiovascular disease, including insulin resistance, hyperlipidemia, obesity, and blood pressure in subjects treated with semaglutide, cilofexor/firsocostat, or their combination versus placebo;
relative change from baseline at Week 72 on lipid parameters including, total cholesterol, LDL, and fasting triglycerides in subjects treated with semaglutide, cilofexor/firsocostat, or their combination versus placebo;
change from baseline at Week 72 in PROs in subjects treated with semaglutide, cilofexor/firsocostat, or their combination versus placebo.

Descriptive statistics of biomarker expression and change from baseline are provided at each sampling time by dose group. Point estimates and 95% confidence intervals may be calculated. Exploratory analyses may also be performed to evaluate the association of individual exploratory biomarkers or combination of biomarkers.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method of treating non-alcoholic steatohepatitis (NASH), comprising administering to a subject with NASH in need of such treatment:
a) semaglutide at a dose of 0.1-3 mg once weekly; and
b) firsocostat at a dose of 15-25 mg once daily.

2. (canceled)

3. (canceled)

4. The method of claim 1, wherein the method comprises administering semaglutide at a dose of 0.24 mg once weekly for four weeks, followed by a dose of 0.50 mg once weekly for four weeks, followed by a dose of 1.0 mg once weekly for four weeks, followed by a dose of 1.7 mg once weekly for four weeks, followed by a dose of 2.4 mg once weekly for at least four weeks, wherein semaglutide is administered by subcutaneous injection.

5. (canceled)

6. The method of claim 4, wherein the method comprises administering firsocostat orally at a dose of 20 mg once daily.

7. (canceled)

8. The method of claim 4, wherein the method further comprises administering cilofexor orally at a dose of 20-120 mg once daily.

9. The method of claim 8, wherein the method comprises administering cilofexor orally at a dose of 30 mg once daily or 100 mg once daily.

10. The method of claim 4, wherein the method comprises administering cilofexor orally at a dose of 30 mg and firsocostat orally at a dose of 20 mg, or wherein the method comprises administering cilofexor orally at a dose of 100 mg and firsocostat orally at a dose of 20 mg.

11. (canceled)

12. (canceled)

13. (canceled)

14. (canceled)

15. The method of claim 4, wherein the method comprises treating the subject for at least 24 weeks, at least 36 weeks, at least 48 weeks, at least 60 weeks, at least 72 weeks, at least 84 weeks, at least 96 weeks, or longer.

16. The method of claim 4, wherein:
a) the subject showed signs of fibrosis prior to treatment;
b) the subject had a FibroTest® score of <0.75 prior to treatment;
c) the subject had ≥10% steatosis prior to treatment, wherein steatosis was determined by MRI-PDFF;
d) the subject had liver stiffness ≥7 kPa prior to treatment wherein liver stiffness was determined by FibroScan®; or
e) wherein the subject was diagnosed with NASH prior to treatment; and/or
f) the subject has type 2 diabetes mellitus.

17.-24. (canceled)

25. The method of claim 4, wherein the subject has one or more of the following laboratory parameters at a baseline timepoint prior to treatment:
a) alanine aminotransferase (ALT) level≤5× the upper limit of normal (ULN);
b) estimated glomerular filtration rate (eGFR)≥30 mL/min;
c) HbA1c≤9.5%;
d) Serum fructosamine≤381 μmol;
e) INR≤1.2;
f) platelet count≥100,000/μL;
g) total bilirubin<1.3× upper limit of normal (ULN); and/or
h) calcitonin≤100 ng/L.

26. (canceled)

27. The method of claim 4, wherein:
a) eGFR is improved by at least 20 mL/min following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to eGFR at a baseline timepoint prior to treatment;
b) steatosis is decreased by at least 5%, at least 10%, or at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to steatosis at a baseline timepoint prior to treatment;
c) median relative MRI-PDFF is reduced by at least 30%, at least 40%, or at least 50% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to MRI-PDFF at a baseline timepoint prior to treatment;
d) liver stiffness is decreased by at least 25% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to liver stiffness at a baseline timepoint prior to treatment;
e) at least one laboratory parameter selected from alanine aminotransferase (ALT), aspartate aminotransferase (AST), bilirubin, gamma-glutamyl transpeptidase (GGT), and alkaline phosphatase (ALP) is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective laboratory parameter at a baseline timepoint prior to treatment;
f) the subject's liver fibrosis is decreased by at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to liver fibrosis at a baseline timepoint prior to treatment;
g) the subject's triglyceride level, LDL cholesterol level, total cholesterol level, HbA_{1c}, fasting plasma glucose level, fasting insulin level, and/or HOMA-IR is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective level at a baseline timepoint prior to treatment;
h) the subject's body weight is decreased by at least 5% following treatment for at least 24 weeks, at least 36 weeks, at least 48 weeks, at least 60 weeks, at least 72 weeks, at least 84 weeks, at least 96 weeks, or longer, compared to the subject's body weight at a baseline timepoint prior to treatment; and/or
i) the subject's FAST score is decreased by at least 0.1, at least 0.2, or at least 0.3 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the FAST score at a baseline timepoint prior to treatment.

28.-40. (canceled)

41. A method of treating non-alcoholic steatohepatitis (NASH), comprising administering to a subject with NASH in need of such treatment:
a) semaglutide at a dose of 0.1-3 mg once weekly; and
b) cilofexor at a dose of 20-120 mg once daily.

42. (canceled)

43. (canceled)

44. The method of claim 41, wherein the method comprises administering semaglutide at a dose of 0.24 mg once weekly for four weeks, followed by a dose of 0.50 mg once weekly for four weeks, followed by a dose of 1.0 mg once weekly for four weeks, followed by a dose of 1.7 mg once weekly for four weeks, followed by a dose of 2.4 mg once weekly for at least four weeks, wherein semaglutide is administered by subcutaneous injection.

45. (canceled)

46. The method of claim 44, wherein the method comprises administering cilofexor orally at a dose of 30 mg once daily or 100 mg once daily.

47. (canceled)

48. (canceled)

49. The method of claim 44, wherein the method comprises treating the subject for at least 24 weeks, at least 36 weeks, at least 48 weeks, at least 60 weeks, at least 72 weeks, at least 84 weeks, at least 96 weeks, or longer.

50. The method of claim 44, wherein:
a) the subject showed signs of fibrosis prior to treatment;
b) the subject had a FibroTest® score of <0.75 prior to treatment;
c) the subject had ≥10% steatosis prior to treatment, wherein steatosis was determined by MRI-PDFF;
d) the subject had liver stiffness ≥7 kPa prior to treatment, wherein liver stiffness was determined by FibroScan®; or
e) wherein the subject was diagnosed with NASH prior to treatment; and/or
f) the subject has type 2 diabetes mellitus.

51.-58. (canceled)

59. The method of claim 44, wherein the subject has one or more of the following laboratory parameters at a baseline timepoint prior to treatment:
a) alanine aminotransferase (ALT) level≤5× the upper limit of normal (ULN);
b) estimated glomerular filtration rate (eGFR)≥30 mL/min;
c) HbA1c≤9.5%;
d) Serum fructosamine≤381 μmol;
e) INR≤1.2;
f) platelet count≥100,000/μL;
g) total bilirubin<1.3× upper limit of normal (ULN); and/or
h) calcitonin≤100 ng/L.

60. (canceled)

61. The method of claim 44, wherein:
a) eGFR is improved by at least 20 mL/min following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to eGFR at a baseline timepoint prior to treatment;
b) steatosis is decreased by at least 5%, at least 10%, or at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to steatosis at a baseline timepoint prior to treatment;
c) median relative MRI-PDFF is reduced by at least 30%, at least 40%, or at least 50% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to MRI-PDFF at a baseline timepoint prior to treatment;
d) liver stiffness is decreased by at least 25% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to liver stiffness at a baseline timepoint prior to treatment;
e) at least one laboratory parameter selected from alanine aminotransferase (ALT), aspartate aminotransferase (AST), bilirubin, gamma-glutamyl transpeptidase (GGT), and alkaline phosphatase (ALP) is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective laboratory parameter at a baseline timepoint prior to treatment;
f) the subject's liver fibrosis is decreased by at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to liver fibrosis at a baseline timepoint prior to treatment;
g) the subject's triglyceride level, LDL cholesterol level, total cholesterol level, HbA_{1c}, fasting plasma glucose level, fasting insulin level, and/or HOMA-IR is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective level at a baseline timepoint prior to treatment;
h) the subject's body weight is decreased by at least 5% following treatment for at least 24 weeks, at least 36 weeks, at least 48 weeks, at least 60 weeks, at least 72 weeks, at least 84 weeks, at least 96 weeks, or longer, compared to the subject's body weight at a baseline timepoint prior to treatment; and/or
i) the subject's FAST score is decreased by at least 0.1, at least 0.2, or at least 0.3 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the FAST score at a baseline timepoint prior to treatment.

62.-74. (canceled)

75. A method of treating non-alcoholic steatohepatitis (NASH), comprising administering to a subject with NASH in need of such treatment:
a) semaglutide at a dose of 0.1-3 mg once weekly;
b) firsocostat orally at a dose of 20 mg once daily; and
c) cilofexor orally at a dose of 30 mg once daily.

76. (canceled)

77. (canceled)

78. The method of claim 75, wherein the method comprises administering semaglutide at a dose of 0.24 mg once weekly for four weeks, followed by a dose of 0.50 mg once weekly for four weeks, followed by a dose of 1.0 mg once weekly for four weeks, followed by a dose of 1.7 mg once weekly for four weeks, followed by a dose of 2.4 mg once weekly for at least four weeks, wherein semaglutide is administered by subcutaneous injection.

79. (canceled)

80. (canceled)

81. (canceled)

82. The method of claim 78, wherein cilofexor and firsocostat are provided in a combined solid dosage form.

83. The method of claim 78, wherein the method comprises treating the subject for at least 24 weeks, at least 36 weeks, at least 48 weeks, at least 60 weeks, at least 72 weeks, at least 84 weeks, at least 96 weeks, or longer.

84. The method of claim 78, wherein:
a) the subject showed signs of fibrosis prior to treatment;
b) the subject had a FibroTest® score of <0.75 prior to treatment;
c) the subject had ≥10% steatosis prior to treatment, wherein steatosis was determined by MRI-PDFF;
d) the subject had liver stiffness ≥7 kPa prior to treatment, wherein liver stiffness was determined by FibroScan®; or
e) wherein the subject was diagnosed with NASH prior to treatment; and/or
f) the subject has type 2 diabetes mellitus.

85.-94. (canceled)

95. The method of claim 78, wherein the subject has one or more of the following laboratory parameters at a baseline timepoint prior to treatment:
a) alanine aminotransferase (ALT) level≤5× the upper limit of normal (ULN);
b) estimated glomerular filtration rate (eGFR)≥30 mL/min;
c) HbA1c≤10%;
d) Serum fructosamine≤400 μmol;
e) INR≤1.4;
f) platelet count≥125,000/μL;
g) total bilirubin<1.3× upper limit of normal (ULN); and/or
h) serum triglyceride level≤250 mg/dL.

96. (canceled)

97. The method of claim 78, wherein:
a) eGFR is improved by at least 20 mL/min following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to eGFR at a baseline timepoint prior to treatment;
b) steatosis is decreased by at least 5%, at least 10%, or at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to steatosis at a baseline timepoint prior to treatment;
c) median relative MRI-PDFF is reduced by at least 30%, at least 40%, or at least 50% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to MRI-PDFF at a baseline timepoint prior to treatment;
d) liver stiffness is decreased by at least 25% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to liver stiffness at a baseline timepoint prior to treatment;
e) at least one laboratory parameter selected from alanine aminotransferase (ALT), aspartate aminotransferase (AST), bilirubin, gamma-glutamyl transpeptidase (GGT), and alkaline phosphatase (ALP) is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective laboratory parameter at a baseline timepoint prior to treatment;
f) the subject's liver fibrosis is decreased by at least 20% following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to liver fibrosis at a baseline timepoint prior to treatment;
g) the subject's triglyceride level, LDL cholesterol level, total cholesterol level, HbA_{1c}, fasting plasma glucose level, fasting insulin level, and/or HOMA-IR is decreased following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, or at least 96 weeks, compared to the respective level at a baseline timepoint prior to treatment;
h) the subject's body weight is decreased by at least 5% following treatment for at least 24 weeks, at least 36 weeks, at least 48 weeks, at least 60 weeks, at least 72 weeks, at least 84 weeks, at least 96 weeks, or longer, compared to the subject's body weight at a baseline timepoint prior to treatment; and/or
i) the subject's FAST score is decreased by at least 0.1, at least 0.2, or at least 0.3 following treatment for at least 10 weeks, at least 12 weeks, at least 16 weeks, at least 20 weeks, at least 24 weeks, at least 36 weeks, at least 48 weeks, at least 60 weeks, at least 72 weeks, at least 84 weeks, at least 96 weeks, or longer, compared to the FAST score at a baseline timepoint prior to treatment.

98.-110. (canceled)
